SEQUENCE LISTING (19 sequences)

SEQ ID NO: 1 - protein, 1372 aa, Artificial Sequence
  Extracellular domain of PLA2R1 (only the part to which autoantibodies bind, i.e. excluding signal peptide, transmembrane domain and intracellular domain)
ALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHS

SEQ ID NO: 2 - protein, 129 aa, Artificial Sequence
  CysR domain (only the part to which autoantibodies bind)
QDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHK

SEQ ID NO: 3 - protein, 154 aa, Artificial Sequence
  CTLD1 domain (only the part to which autoantibodies bind)
PTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYA

SEQ ID NO: 4 - protein, 161 aa, Artificial Sequence
  CTLD5 domain (only the part to which autoantibodies bind)
KIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGT

SEQ ID NO: 5 - protein, 150 aa, Artificial Sequence
  CTLD7 domain (only the part to which autoantibodies bind)
EKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPEL

SEQ ID NO: 6 - protein, 163 aa, Artificial Sequence
  CTLD8 (P1235-S1397)
PPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHS

SEQ ID NO: 7 - protein, 229 aa, Artificial Sequence
  Delta7 (P1235-Q1463: CTLD8 domain plus transmembrane and intracellular domains)
PPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSIIPLAVVLTLIVIVAICTLSFCIYKHNGGFFRRLAGFRNPYYPATNFSTVYLEENILISDLEKSDQ

SEQ ID NO: 8 - protein, 324 aa, Artificial Sequence
  CysR-FnII-CTLD1 domain (only the part to which autoantibodies bind)
QDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLN

SEQ ID NO: 9 - protein, 1038 aa, Artificial Sequence
  CTLD2-8 domain (only the part to which autoantibodies bind)
HIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHS

SEQ ID NO: 10 - protein, 1437 aa, Artificial Sequence
  pla2r extracellular domain with His flag and HA tags, as used in the examples)
MLLSPSLLLLLLLGAPRGCAEGVAAALTPERLLEWHHHHHHGASDYKDHDGDYKDHDIDYKDDDDKQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSYPYDVPDYA

SEQ ID NO: 11 - protein, 204 aa, Artificial Sequence
  CysR with His flag and HA tags, as used in the examples
MLLSPSLLLLLLLGAPRGCAEGVAAALTPERLLEWHHHHHHGASDYKDHDGDYKDHDIDYKDDDDKQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKYPYDVPDYA

SEQ ID NO: 12 - protein, 229 aa, Artificial Sequence
  CTLD1 with His flag and HA tags, as used in the examples
MLLSPSLLLLLLLGAPRGCAEGVAAALTPERLLEWHHHHHHGASDYKDHDGDYKDHDIDYKDDDDKPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYAYPYDVPDYA

SEQ ID NO: 13 - protein, 236 aa, Artificial Sequence
  CTLD5 with His flag and HA tags, as used in the examples
MLLSPSLLLLLLLGAPRGCAEGVAAALTPERLLEWHHHHHHGASDYKDHDGDYKDHDIDYKDDDDKKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTYPYDVPDYA

SEQ ID NO: 14 - protein, 225 aa, Artificial Sequence
  CTLD7 with His flag and HA tags, as used in the examples
MLLSPSLLLLLLLGAPRGCAEGVAAALTPERLLEWHHHHHHGASDYKDHDGDYKDHDIDYKDDDDKEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELYPYDVPDYA

SEQ ID NO: 15 - protein, 238 aa, Artificial Sequence
  CTLD8 with His flag and HA tags, as used in the examples
MLLSPSLLLLLLLGAPRGCAEGVAAALTPERLLEWHHHHHHGASDYKDHDGDYKDHDIDYKDDDDKPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSYPYDVPDYA

SEQ ID NO: 16 - protein, 304 aa, Artificial Sequence
  Delta7-HA with His flag and HA tags, as used in the examples
MLLSPSLLLLLLLGAPRGCAEGVAAALTPERLLEWHHHHHHGASDYKDHDGDYKDHDIDYKDDDDKPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSIIPLAVVLTLIVIVAICTLSFCIYKHNGGFFRRLAGFRNPYYPATNFSTVYLEENILISDLEKSDQYPYDVPDYA

SEQ ID NO: 17 - protein, 399 aa, Artificial Sequence
  CysR-FnII-CTLD1 with His flag and HA tags, as used in the examples
MLLSPSLLLLLLLGAPRGCAEGVAAALTPERLLEWHHHHHHGASDYKDHDGDYKDHDIDYKDDDDKQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNYPYDVPDYA

SEQ ID NO: 18 - protein, 1113 aa, Artificial Sequence
  CTLD2 to CTLD8 with His flag and HA tags, as used in the examples
MLLSPSLLLLLLLGAPRGCAEGVAAALTPERLLEWHHHHHHGASDYKDHDGDYKDHDIDYKDDDDKHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSYPYDVPDYA

SEQ ID NO: 19 - protein, 1463 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWMGLNQLDEHAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGNNETIKWFDGTPTDQSNWGIRKPDTDYFKPHHCVALRIPEGLWQLSPCQEKKGFICKMEADIHTAEALPEKGPSHSIIPLAVVLTLIVIVAICTLSFCIYKHNGGFFRRLAGFRNPYYPATNFSTVYLEENILISDLEKSDQ

(19) United States
(12) Patent Application Publication
(10) Pub. No.: US 2023/0204602 A1
(43) Pub. Date: Jun. 29, 2023

(54) PROFILING OF IMMUNODOMINANT PLA2R1 EPITOPES AS A PROGNOSIS AND PREDECTIVE FACTOR IN MEMBRANOUS NEPHROPATHY

(71) Applicants: CENTRE NATIONAL DE LA RECHERCHE SCIENTIFIQUE - CNRS -, Paris (FR); UNIVERSITE COTE D'AZUR, Nice Cedex 2 (FR)

(72) Inventors: Joana JUSTINO, Valbenno (FR); Gérard LAMBEAU, Cabris (FR)

(21) Appl. No.: 17/998,851

(22) PCT Filed: May 17, 2021
(86) PCT No.: PCT/EP2021/062960

§ 371 Date: Nov. 15, 2022

(30) Foreign Application Priority Data
May 15, 2020 (EP) 20305501.7

(51) Int. Cl. G01N 33/68

(52) U.S. Cl. CPC G01N 33/6893; G01N 2800/52; G01N 2800/347

(57) ABSTRACT

The invention relates to a method for predicting the prognosis of a patient suffering from membranous nephropathy and for determining the likelihood of response to immunosuppressive treatment, based on the analysis of PLA2R1 epitope immunodomi-nance profiling.

### FIELD OF THE INVENTION

The invention relates to a method for predicting the prognosis of a patient suffering from membranous nephropathy and for determining the likelihood of response to immunosuppressive treatment, based on the analysis of PLA2R1 epitope immunodominance profiling.

### BACKGROUND OF THE INVENTION

Membranous Nephropathy (MN) is a rare but severe autoimmune kidney disease. It is a common cause of nephrotic syndrome in adults, affecting both native and transplanted kidneys (Ronco, P, Debiec, H: Pathophysiological advances in membranous nephropathy: time for a shift in patient’s care. Lancet, 385: 1983-1992, 2015). The clinical evolution is complex, ranging from spontaneous remission to persistent proteinuria and end-stage kidney disease (ESKD).

In 2009, Beck and colleagues identified the M-type phospholipase A2 receptor (PLA2R1) as the major autoantigen in MN, with approximately 70% of patients having circulating anti-PLA2R1 autoantibodies, associated with in situ immune complex deposits in the glomerulus, podocyte injury and high proteinuria (Beck, LH, Jr., Bonegio, RG, Lambeau, G, Beck, DM, Powell, DW, Cummins, TD, Klein, JB, Salant, DJ: M-type phospholipase A2 receptor as target antigen in idiopathic membranous nephropathy. N Engl J Med, 361: 11-21, 2009).

PLA2R1 is a 180 kDa type I transmembrane glycoprotein consisting of a large extracellular region comprising 10 individual domains linked by short linker sequences: a N-terminal cysteine-rich domain (CysR), a fibronectin type II domain (FnII) and eight distinct C-type lectin-like domains (CTLDs).

Standardized assays, such as IIFT (indirect immunofluorescence test), ELISA (enzyme-linked immunosorbent assay) and ChLIA (chemiluminescence immunoassay), can be used to detect anti-PLA2R1 autoantibodies, and are now used in clinical practice for PLA2R1-associated MN diagnosis, prognosis and theragnosis (IIFT: E. Hoxha, S. Harendza, G. Zahner, U. Panzer, O. Steinmetz, K. Fechner, U. Helmchen, R.A. Stahl, “An immunofluorescence test for phospholipase-A2-receptor antibodies and its clinical usefulness in patients with membranous glomerulonephritis”, Nephrol Dial Transplant, 26 (2011) 2526-2532; ELISA: Dahnrich, C, Komorowski, L, Probst, C, Seitz-Polski, B, Esnault, V, Wetzels, JF, Hofstra, JM, Hoxha, E, Stahl, RA, Lambeau, G, Stocker, W, Schlumberger, W: “Development of a standardized ELISA for the determination of autoantibodies against human M-type phospholipase A2 receptor in primary membranous nephropathy”. Clin Chim Acta, 421C: 213-218, 2013; ChLIA: C. Dähnrich, S. Saschenbrecker, I. Gunnarsson, W. Schlumberger, P. Ronco, H. Debiec, “Development of a Standardized Chemiluminescence Immunoassay for the Detection of Autoantibodies Against Human M-Type Phospholipase A2 Receptor in Primary Membranous Nephropathy”, Kidney international reports, 5 (2020) 182-188; and). Anti-PLA2R1 titer helps to predict clinical outcome, with low titer associated with spontaneous remission, and high titer associated with progression to severe disease and ESKD.

Furthermore, several studies have demonstrated that anti-PLA2R1 autoantibodies recognize multiple conformational epitopes in PLA2R1. Seitz-Polski&Dolla and colleagues identified CysR, CTLD1 and CTLD7 as three distinct epitope-containing domains that may be reminiscent of a mechanism of epitope spreading (Seitz-Polski, B, Dolla, G, Payre, C, Girard, CA, Polidori, J, Zorzi, K, Birgy-Barelli, E, Jullien, P, Courivaud, C, Krummel, T, Benzaken, S, Bernard, G, Burtey, S, Mariat, C, Esnault, VL, Lambeau, G: Epitope Spreading of Autoantibody Response to PLA2R Associates with Poor Prognosis in Membranous Nephropathy. J Am Soc Nephrol, 27: 1517-1533, 2016). All patients were found to be CysR-positive while only a subset was positive for CTLD1 and/or CTLD7.

Thus, according to profiling by epitope positivity or not towards the different epitope-containing domains, WO2017/009245 describes the stratification of patients into two main subgroups: those with single positivity against the CysR domain of PLA2R1, and those with multiple positivities against both CysR and CTLD1 and/or CTLD7 domains. This stratification allows the classification of patients in two categories: good prognosis/good responders to treatment and poor prognosis/poor responders to treatment. Indeed, a patient presenting autoantibodies directed only against the CysR domain of PLA2R1 will be of good prognosis. On the contrary, a patient presenting circulating autoantibodies directed against CysR but also CTLD1 and/or CTLD7 of PLA2R1 will be of poor prognosis (Seitz-Polski, B, Debiec, H, Rousseau, A, Dahan, K, Zaghrini, C, Payre, C, Esnault, VLM, Lambeau, G, Ronco, P: Phospholipase A2 Receptor 1 Epitope Spreading at Baseline Predicts Reduced Likelihood of Remission of Membranous Nephropathy. J Am Soc Nephrol, 29: 401-408, 2018).

Overall, the above data indicate that anti-PLA2R1 titer and profiling based on epitope positivity help to predict response to immunosuppressive therapy including treatment with rituximab. This contrasts with the recent conclusions from Reinhard et al., suggesting that anti-PLA2R1 titer but not epitope positivity can predict clinical outcome. The discrepant conclusions may be due to several factors, particularly the type of immunosuppressive treatment, which was mainly based on alkylating drugs and/or calcineurin inhibitors given to most patients in the Reinhard’s cohort (Reinhard, L, Zahner, G, Menzel, S, Koch-Nolte, F, Stahl, RAK, Hoxha, E: Clinical Relevance of Domain-Specific Phospholipase A2 Receptor 1 Antibody Levels in Patients with Membranous Nephropathy. J Am Soc Nephrol, 2019). In fact, it has recently been shown that immunosuppressive treatment with alkylating drugs such as cyclophosphamide induced a faster and stronger disappearance of anti-PLA2R1 antibodies than rituximab (Van de Logt, AE, Dahan, K, Rousseau, A, van der Molen, R, Debiec, H, Ronco, P, Wetzels, J: Immunological remission in PLA2R-antibody-associated membranous nephropathy: cyclophosphamide versus rituximab. Kidney Int, 93: 1016-1017, 2018), which may overcome subtle differences of the humoral response between patients and hence the predictive values of the different anti-PLA2R1 features (titer versus epitope profile).

Despite the above studies to better diagnose and predict outcome in patients based on anti-PLA2R1 titer or epitope positivity, treatment options with conservative treatment or various immunosuppressants are not well defined based on these anti-PLA2R1 features. More specifically, it is necessary to establish cut-off values of anti-PLA2R1 titer or identify new biomarkers with added value over titer or epitope positivity that would help to guide treatment options with immunosuppressants and predict the likelihood of a response.

At the molecular level, despite the above advances in the identification of PLA2R1 epitopes, we lack a complete description of the anti-PLA2R1 humoral autoimmune response observed among patients, including the identification of the total number of epitopes and of those most prevalent and immunodominant. This detailed description may lead to the discovery of new molecular biomarkers to identify patients at risk of poor clinical outcome and to monitor the efficacy of therapy, in a more sensitive and refined way as compared to the above anti-PLA2R1 features consisting of anti-PLA2R1 titer and epitope positivity.

### SUMMARY OF THE INVENTION

The inventors now showed that the anti-PLA2R1 humoral response observed among patients at diagnosis is in most cases polyclonal but variable, with the presence of multiple and new autoantibodies differing in prevalence and immunodominance, allowing a novel profiling of patients based on those features.

The inventors further demonstrate that immunodominance profiling is clinically useful to stratify patients and guide treatment options more precisely than methods described in the state of the art.

The inventors have first clearly demonstrated that the CysR and/or CTLD1 domains of PLA2R1 contain the key independent immunodominant epitopes that drive the humoral response for most patients, while other PLA2R1 epitope-containing domains play a minor role in the strength of the autoimmune response.

The inventors further showed that profiling of patients based on immunodominance features, for instance as measured by either direct or competitive ELISA assays, led to the stratification of patients into two groups, according to the nature of the autoantibody which mostly drives the humoral response:
patients immunodominant for CTLD1 (iC1), when the humoral response to PLA2R1 is mostly driven by anti-CTLD1 autoantibodies and
patients non immunodominant for CTLD1, when the humoral response to PLA2R1 is not mostly driven by anti-CTLD1 autoantibodies.

The inventors also demonstrate that immunodominance can be used as a clinical biomarker per se and also as an additional clinical biomarker which can be optionally combined with anti-PLA2R1 titer to refine clinical outcome and the likelihood of response to treatment.

Furthermore, the inventors demonstrated that immunodominance can be accurately assessed by two methods:

1. Either by a competition assay (such as for example a competition ELISA assay, a chemiluminescence immunoassay ChLIA, or immunofluorescence assay), in which case the patients non-immunodominant for CTLD1 can be optionally stratified in two subgroups:
immunodominant CysR (iCR) patients whose humoral response to PLA2R1 is mostly driven by anti-CysR autoantibodies;
non-immunodominant (non-iDom) patients whose humoral response to PLA2R1 is driven by neither anti-CysR nor anti-CTLD1 nor any other anti-PLA2R1 autoantibodies.

2. Either by measuring the ratio of anti-CTLD1 autoantibody titer vs. anti-CysR autoantibody titer (or inversely). In this case the value of the ratio can be compared to a reference value (for instance a median value of ratios measured for MN patients from a cohort). In this embodiment patients can be stratified as iC1 (CTLD 1 immunodominance) or iCR (CysR immunodominance).

Stratifying patients according to immunodominance can be useful to guide and treat patients with different immunosuppressants, in particular rituximab, for a better likelihood of response to treatment.

Indeed, the immunodominance profiling according to the invention allows identifying patients with a good prognosis (iCR, and optionally non-iDom patients if immunodominance is assessed by a competition assay) from patients with a poor prognosis (iC1 patients). From a pathophysiological point of view, iC1 patients differ from iCR (and optionally non-iDom patients if immunodominance is assessed by a competition assay) patients by exhibiting two classes of immunodominant autoantibodies targeting PLA2R1 on both CysR and CTLD1 domains, which may exert a synergistic effect and may be more pathogenic at inducing podocyte injury and heavy proteinuria.

The immunodominance profiling allows identifying and stratifying patients in a more sensitive way. Indeed, the immunodominance profiling according to the invention allows identifying patients with a good prognosis and likelihood of response to treatment which would have not been considered as good responders by methods previously described in the state of the art such as anti-PLA2R1 titer and profiling based on epitope positivity. The method according to the invention also permits distinction between the new iCR and iC1 groups in terms of prognosis but also in terms of response to treatment and avoid false positives/negatives.

Thus, in a first aspect, the invention relates to a method for determining PLA2R1 immunodominance in a sample obtained from a patient suffering from membranous nephropathy (MN), preferably idiopathic membranous nephropathy (iMN), and more preferably PLA2R1-associated membranous nephropathy (PLA2R1-MN), comprising a step of determining the nature of the immunodominant antibody which mostly drives the humoral response in said sample. In one embodiment, this is performed by various types of competition assays (such as ELISA, ChLIA, immunofluorescence, etc) between PLA2R1 and saturating amount of a CysR-FnII-CTLD1 fragment, preferably with saturating amount of polypeptide competitors selected among a CysR fragment, a CTLD1 fragment and CysR-FnII-CTLD1 or CTLD2-8 fragments, preferably a CysR fragment, a CTLD1 fragment, a CysR-FnII-CTLD1 fragment, a CTLD5 fragment, and a CTLD7 fragment or a mixture thereof. In a second embodiment, PLA2R1 immunodominance can be determined by measuring anti-CTLD1 and anti-CysR titers and calculating the ratio of those titers (See details below). Preferably the ratio of titers is used to achieve the purpose of any of the methods according to the present invention.

The inventors showed that in PLA2R1-positive membranous nephropathy patients, circulating anti-PLA2R1 autoantibodies can recognize up to 5 epitope-containing domains, including CysR, CTLD1, CTLD5, CTLD7 and CTLD8.

They advantageously demonstrated that the two N-terminal CysR and CTLD1 domains harbor the major immunodominant epitopes which contribute to most of the anti-PLA2R1 titer measured by a standardized commercially-available ELISA assay (see Dahnrich, C, Komorowski, L, Probst, C, Seitz-Polski, B, Esnault, V, Wetzels, JF, Hofstra, JM, Hoxha, E, Stahl, RA, Lambeau, G, Stocker, W, Schlumberger, W: “Development of a standardized ELISA for the determination of autoantibodies against human M-type phospholipase A2 receptor in primary membranous nephropathy”. Clin Chim Acta, 421C: 213-218, 2013). Conversely, the C-terminal domains CTLD5, CTLD7 and CTLD8 harbor non-immunodominant epitopes which collectively contribute to little of the anti-PLA2R1 titer.

Together, the overall humoral autoimmune response appears to be mostly driven by the N-terminal CysR and/or CTLD1 domains functioning as two key immunodominant epitope-containing domains while the distal positivity to the C-terminal domains contributes little to the anti-PLA2R1 titer.

The inventors demonstrate that the consideration of immunodominance in either three groups (iCR/non-iDom vs. iC1, when immunodominance is assessed by a competition assay) or two groups (iCR vs. iC1, when considering the ratio of anti-CTLD1 vs. anti-CysR titer for a patient) of patients can accurately predict clinical outcome. Indeed, as compared to previous known methods typically based on anti-PLA2R1 titer or epitope positivity, the present methods stratify differently the patients (see notably FIGS. 6-9) and allow a better identification of patients at risk.

In a second aspect, the invention relates to a method of predicting the prognosis of a patient suffering from membranous nephropathy comprising determining PLA2R1 immunodominance in a sample obtained from said patient according to the method for determining PLA2R1 immunodominance, wherein:
a patient immunodominant for CTLD1 exhibits a poor prognosis;
a patient non immunodominant for CTLD1, typically a patient immunodominant for CysR (and/or a patient non-immunodominant if immunodominance is assessed through a competition assay) exhibits a good prognosis.

In a third aspect, as the inventors also showed that the immunodominance profiling helps to guide treatment with immunosuppressants and predicts the likelihood of a response, the invention relates to a method of predicting the response to an immunosuppressive treatment of a patient suffering from membranous nephropathy comprising determining PLA2R1 immunodominance in a sample obtained from said patient according to the method for determining PLA2R1 immunodominance, wherein:
a patient immunodominant for CTLD1 is resistant to treatment with immunosuppressants;
a patient non-immunodominant for CTLD1, typically who is immunodominant for CysR (and/or a patient non-immunodominant, if immunodominance is measured through a competition assay) is a good responder to immunosuppressants.

In a fourth aspect, the invention relates to a method for the treatment of membranous nephropathy in a subject in need thereof comprising:
determining PLA2R1 immunodominance in a sample obtained from a patient suffering from membranous nephropathy by determining the nature of the antibody which mostly drives the humoral response in said sample according to a method for determining PLA2R1 immunodominance, and:
administering an effective amount of a symptomatic treatment or an effective amount of an immunosuppressant to said patient, when said patient is immunodominant for CysR or is non-immunodominant, and thus considered as a good responder to said treatment;
repeating the effective amount of the immunosuppressant or administering an effective amount of an alternative or combined stronger immunosuppressive therapy, or initiating a hemodialysis, when said patient is immunodominant for CTLD1 and is thus resistant to said immunosuppressant.

The present invention also encompasses a kit comprising:
means for detecting an autoantibody binding to CysR, CTLD1 or the extracellular domain of PLA2R1, preferably a secondary antibody binding to human IgG class autoantibodies, more preferably carrying a detectable label, and either
   an immobilized polypeptide comprising CysR or a variant thereof binding to autoantibodies to CysR,
   an immobilized polypeptide comprising CTLD1 or a variant thereof binding to autoantibodies to CTLD1 and
   one or more polypeptides, preferably all, in an immobilized form, from the group comprising CTLD5, CTLD7 and CTLD8 or a variant thereof binding to autoantibodies to CTLD5, CTLD7 or CTLD8, respectively,
   an immobilized polypeptide comprising the extracellular domain of PLA2R1 or a variant thereof binding to autoantibodies to the extracellular domain of PLA2R1 and
   optionally a negative control or cut-off indicator indicating unspecific binding of autoantibodies if present and
wherein the immobilized polypeptides are immobilized on one or more diagnostically useful carriers spatially separated such that an antibody bound to one of the polypeptides can be distinguished from an autoantibody bound to any of the other polypeptides. or
   an immobilized polypeptide comprising the extracellular domain of PLA2R1 or a variant thereof binding to autoantibodies to the extracellular domain of PLA2R1 and
   a non-immobilized polypeptide comprising CysR or a variant thereof binding to autoantibodies to CysR and
   a non-immobilized polypeptide comprising CTLD1 or a variant thereof binding to autoantibodies to CTLD1 and
   one or more polypeptides, preferably all, in a non-immobilized form, from the group comprising CTLD5, CTLD7 and CTLD8 or a variant thereof binding to autoantibodies to CTLD5, CTLD7 or CTLD8, respectively,
   optionally a negative control or cut-off indicator indicating unspecific binding of autoantibodies if present,
wherein the kit preferably comprises in addition a first control comprising an antibody to CysR and a second control comprising an antibody to CTLD1, more preferably in addition a control comprising an antibody to CTLD5, a control comprising an antibody to CTLD7 and a control comprising an antibody to CTLD8.

### FIGURES

FIG. 1 shows the prevalence and epitope profile in a cohort of 142 PLA2R1-positive MN patients. (FIG. 1A) Screening of 142 PLA2R1-positive patients by ELISA (IgG4 detection) with the 10 individual domains of PLA2R1 shows reactivity against 5 domains with decreasing prevalence: CysR (CR), CTLD5 (C5), CTLD1 (C1), CTLD7 (C7) and CTLD8 (C8). None of the five remaining domains were recognized. (FIG. 1B) Patients’ reactivity to single domains can be combined to provide different epitope profiles among which CRC5, CRC1C5C7, CRC1, CR, CRC5C7 and CRC1C5 are the most prevalent.

FIG. 2 represents the relationship between epitope positivity and anti-PLA2R1 titer. (FIG. 2A) Patients (n=142) were ranked by increasing anti-PLA2R1 titer (standardized ELISA, total IgG detection) and positivity for the different domains was superimposed to display the different patterns. All patients were positive for CysR. In the first tertile, 29.8% of patients were CTLD1, 53.2% CTLD5, 8.5% CTLD7 and 0% CTLD8. In the second tertile, 35.4% of patients were CTLD1, 75.0% CTLD5, 35.4% CTLD7 and 4.2% CTLD8. In the third tertile, 74.5% of patients were CTLD1, 68.1% CTLD5, 66.0% CTLD7 and 6.4% CTLD8.

(FIG. 2B) Patients (n=142) were ranked according to the complexity of their epitope positivity from the CysR domain to the C-terminal end of PLA2R1 and positivity or not for CTLD1. The increased complexity of epitope positivity appears to be associated with anti-PLA2R1 titer.

FIG. 3 demonstrates the relationship between immunodominance profile and anti-PLA2R1 titer. (FIG. 3A) Patients (n=136) were ranked by increasing anti-PLA2R1 titer (standardized ELISA, total IgG detection) and their immunodominance was displayed. In the first tertile, 66.7% of patients were iCR, 24.4% iC1 and 8.9% non-iDom. In the second tertile, 60.9% of patients were iCR, 21.7% iC1 and 17.4% non-iDom. In the third tertile, 37.8% of patients were iCR, 62.2% were iC1 and 0% non-iDom. The stars above titer indicate positivity for CTLD7 and/or CTLD8. (FIG. 3B) Patients (n=136) were ranked according to their immunodominant profile (iCR, non-iDom or iC1) combined with the complexity of their epitope positivity from the CysR domain to the C-terminal region of PLA2R1. CR and C1 are indicated in bold when they become immunodominant. The increased complexity of epitope profiles appears to be associated with anti-PLA2R1 titer for both iCR and iC1 groups. Note that patients from the non-iDom group have relatively low titers, in line with the notion that high titers are observed only when immunodominance to CysR or CTLD1 is effective.

FIG. 4 highlights that CysR and CTLD1 are two immunodominant PLA2R1 domains and define different groups of PLA2R1-positive MN patients. (FIG. 4A) Representative competition assays by in-house ELISA (IgG4 detection) for three patients allowing classification in three distinct groups: iCR, iC1 and non-iDom. Note that the chosen representative patients have the same profile of epitope positivity (CRC1C5C7) but different profiles of immunodominance. (FIG. 4B) Stratification of patients (n=136) according to their immunodominant profile shows that the majority of them (n=75, 55.1%) have CysR as immunodominant PLA2R1 domain (iCR) while a significant number (n=49, 36.0%) have CTLD1 as immunodominant PLA2R1 domain (iC1). A small group of patients (n=12, 8.8%) are classified as non-iDom. The immunodominant profile could not be assigned for 6 patients from the original cohort of 142 patients because of lack of serum.

FIG. 5 describes the correlation between standardized anti-PLA2R1 titer (total IgG detection) and in-house anti-PLA2R1 titer (IgG4 detection), anti-CysR or anti-CTLD1 titers (IgG4 detection) for PLA2R1-positive MN patients overall (n=141) or according to immunodominance (n=135). (FIG. 5, top panels) Correlations between anti-PLA2R1 titer measured by standardized ELISA (total IgG) and in-house ELISA (IgG4). (FIG. 5, middle and bottom panels) Correlations between anti-PLA2R1 titer measured by standardized ELISA (total IgG) and anti-CysR titer (FIG. 5, middle panels) or anti-CTLD1 titer (FIG. 5, bottom panels). Note the weaker correlations between anti-PLA2R1 and anti-CTLD1 titers for the whole population and iCR patients as compared to iC1 patients.

FIG. 6 represents the clinical outcome of patients according to their profile by immunodominance (FIG. 6A), combined epitope positivity restricted or not to CysR (FIG. 6B), single positivity to epitope-containing domains (FIG. 6C), and anti-PLA2R1 titer (FIG. 6D). FIG. 6E further shows the clinical outcome of patients according to their profile of immunodominance when considering only those with an anti-PLA2R1 titer below 200 RU/mL.

FIG. 7 demonstrates the high concordance for the stratification of patients by immunodominance between the methods using competition ELISA and analysis of the anti-CTLD1/anti-CysR ratio. Patients (n=135) were ranked by increasing anti-CTLD1/anti-CysR ratio and their profile of immunodominance, as determined by competition ELISA, was displayed. The data show a high concordance between the two methods. For instance, when considering the median value, 71% of the iCR patients are below the median while 90% of the iC1 patients are above the median. Similarly, 66% of non-iDom patients are below the median, in agreement with their similar clinical features with iCR patients.

FIG. 8 represents the anti-PLA2R1 titer (FIG. 8A, standardized ELISA, detection of total IgG), and anti-CysR (FIG. 8B) and anti-CTLD1 (FIG. 8C) titers (in-house ELISA, IgG4 detection) of patients according to stratification below and above the median (0.0324) of the anti-CTLD1/anti-CysR ratio. No significant difference in anti-PLA2R1 titer is observed below and above the median, arguing that patients below the median, who are mostly iCR, can have similar titers as those above the median, who are mostly iC1. As expected, patients below the median have higher anti-CysR titer while those above the median have higher anti-CTLD1 titer.

FIG. 9 shows that the stratification of patients by immunodominance, as defined by the value of ratio of anti-CTLD1/anti-CysR titer (below and above the median value of 0.0324 in the study cohort) is associated with clinical outcome and response to treatment with rituximab. The clinical outcome of patients as a full population (FIG. 9A) or for those with an anti-PLA2R1 titer below 200 RU/mL (FIG. 9B) is shown according to their immunodominance as defined by the ratio of anti-CTLD1/anti-CysR titers above or below the median value (0.0324).

### DETAILED DESCRIPTION OF THE INVENTION

### Methods for Determining PLA2R1 Immunodominance

The inventors demonstrated that the autoimmune response against PLA2R1 is polyclonal and leads to the presence of multiple circulating anti-PLA2R1 autoantibodies targeting up to five distinct PLA2R1 domains that spread over the entire extracellular region from the N-terminal to the C-terminal ends: CysR, CTLD1, CTLD5, CTLD7 and CTLD8.

The inventors further determined the prevalence and immunodominance properties of the five epitope-containing domains relative to the standardized anti-PLA2R1 titer and in particular determined which PLA2R1 domains contain the major immunodominant epitopes contributing the most to the strength of antibody binding on the full PLA2R1 antigen (full extracellular domain whose the sequence is defined below).

Towards these goals, they screened a cohort of 142 PLA2R1-positive membranous nephropathy (MN) patients against the 10 single PLA2R1 domains by ELISA. The five epitope-containing domains CysR, CTLD1, CTLD5, CTLD7 and CTLD8 were recognized with different prevalence. None of the other single domains, namely FnII, CTLD2, CTLD3, CTLD4 and CTLD6 were recognized. The CysR domain plays a central role and is undoubtedly the major immunodominant epitope-containing domain, based on both its highest prevalence (100%) and its highest contribution to the anti-PLA2R1 signal as measured by competition ELISA (up to 100%). CTLD5 was the second most prevalent domain with 65.5% reactivity followed by CTLD1 (46.5%), CTLD7 (36.6%) and CTLD8 (3.5%).

Beyond prevalence, the inventors determined which PLA2R1 domain contains the major immunodominant epitopes that would contribute the most to the signal measured by ELISA on the full PLA2R1 antigen.

Therefore, in a first aspect, the present invention relates to a method for determining PLA2R1 immunodominance in a sample obtained from a patient suffering from membranous nephropathy.

In one embodiment, this method comprises a step of determining the nature of the antibody which mostly drives the humoral response in said sample by performing competition assays with saturating amount of polypeptide competitors selected among a CysR fragment, a CTLD1 fragment, a CysR-FnII-CTLD1 fragment, or mixture thereof.

In another embodiment, the step of determining the nature of the antibody which mostly drives the humoral response in the sample from the patient is achieved by establishing a ratio between the titers of the anti-CTLD1 antibodies and the anti-CysR antibodies.

The term “membranous nephropathy” (MN) has its general meaning in the art and refers to a renal autoimmune disease which is a frequent cause of adult nephrotic syndrome. It encompasses primary membranous nephropathy, also called “idiopathic membranous nephropathy” and multiple secondary membranous nephropathies that are caused by other diseases such as various cancers and autoimmune diseases or infections including systemic lupus erythematosus, rheumatoid arthritis, hepatitis B or infection by HIV. Idiopathic membranous nephropathy (iMN) or primary MN (pMN) is considered to be an autoimmune disease targeting the kidney glomerulus, with PLA2R1 as the major autoantigen target. Preferably, the major form of iMN or pMN is PLA2R1-associated membranous nephropathy.

The term “PLA2R1” or “PLA2R” or “secretory phospholipase A2 receptor 1” refers to the M-type phospholipase A2 receptor, a receptor in humans that is encoded by the PLA2R1 gene, particularly known as a major autoantigen in idiopathic membranous nephropathy. An exemplary human native PLA2R1 amino acid sequence is provided in NP_001007268 (GenPept database), with various recommended and alternative names and isoforms provided in Q13018 (typically the membrane-bound isoform 1 but also other isoforms such as soluble PLA2R1 isoform 2) (UniProtKB database) and other databases and referred herein as SEQ ID NO:19.

It is however understood that polymorphisms or variants with different sequences exist in various subject genomes. The term PLA2R (or PLA2R1) according to the invention thus encompasses all mammalian variants of PLA2R, and genes that encode this protein with at least 50%, 55%, 60%, 65%, 70%, 75%, 80%, or typically 85%, 90%, or 95%, 99% or 99.5% identical to SEQ ID NO: 19.

The state of the art comprises various methods that may be used to align two given nucleic acid or amino acid sequences and to calculate the degree of identity, see for example Arthur Lesk (2008), Introduction to bioinformatics, Oxford University Press, 2008, 3rd edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007): Clustal W and Clustal X version 2.0. Bioinformatics, 23, 2947-2948) is used applying default settings. As used herein, the expression “percentage of identity” between two sequences, means the percentage of identical bases or amino acids between the two sequences to be compared, obtained with the best alignment of said sequences, this percentage being purely statistical and the differences between these two sequences being randomly spread over the two sequences. As used herein, “best alignment” or “optimal alignment”, means the alignment for which the determined percentage of identity (see below) is the highest. Sequence comparison between two nucleic acid sequences is usually realized by comparing these sequences that have been previously aligned according to the best alignment; this comparison is realized on segments of comparison in order to identify and compared the local regions of similarity. The best sequence alignment to perform comparison can be realized, besides manually, by using the global homology algorithm developed by SMITH and WATERMAN (Ad. App. Math., vol.2, p:482, 1981), by using the local homology algorithm developed by NEDDLEMAN and WUNSCH (J. Mol. Biol, vol.48, p:443, 1970), by using the method of similarities developed by PEARSON and LIPMAN (Proc. Natl. Acd. Sci. USA, vol.85, p:2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar, Robert C, Nucleic Acids Research, vol. 32, p: 1792, 2004). To get the best local alignment, one can preferably use BLAST software. The percentage of identity between two sequences is determined by comparing these two sequences optimally aligned, the sequences being able to comprise additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

The variant may be created by incorporating insertions, mutations, deletions into a wild-type sequence as well as fusing to the N- and/or C-terminus of the wild-type sequence or a variant thereof additional sequences, for example artificial linker and/or tag sequences. In a preferred embodiment, the variant comprises at least 30 successive amino acid residues from or derived from the wild-type sequence. Particularly preferred variants include homologous sequences from animals, preferably mammals.

The variant of the polypeptide has biological activity. In a preferred embodiment, such biological activity is the ability to bind to the antibody to be detected or the level of which is to be determined, from a sample from a patient.

As used herein, the term “subject” or “patient” refers to an individual with symptoms of and/or suspected of suffering from membranous nephropathy. In the context of the invention, the subject or patient is preferably a subject suffering or suspected of suffering from idiopathic membranous nephropathy, preferably PLA2R1-associated MN.

The “sample” is a biological sample obtained from said subject. Such samples include, but are not limited to, bodily fluids which may or may not contain cells, e.g., blood (e.g. whole blood, serum or plasma) or urine. Such samples also include biopsies (for example kidney biopsy). Preferably, said sample is a body fluid of said subject. Non-limiting examples of samples include, but are not limited to, whole blood sample, plasma or serum, or urine. Preferably, said biological sample is serum. The term biological sample also encompasses any material derived by processing a biological sample. Derived materials include, but are not limited to, cells (or their progeny) isolated from the sample, or proteins extracted from the sample like plasma exchange obtained after various types of dialysis techniques to treat chronic kidney failure (hemodialysis, peritoneal dialysis, etc). Processing of a biological sample may involve one or more of: filtration, distillation, extraction, concentration, inactivation of interfering components, addition of reagents, and the like.

In a preferred embodiment, the sample is selected among bodily fluids which may or may not contain cells or biopsies.

In a yet preferred embodiment, the sample is selected among whole blood, serum, plasma, urine or kidney biopsy.

### Competition Assay

In some embodiments, the determination of PLA2R1 immunodominance in a sample obtained from a patient suffering from membranous nephropathy comprises a step of performing a competition assay, typically an ELISA competition assay, between saturating amounts of at least one polypeptide competitor and the full PLA2R1 antigen in a biological sample.

The “full PLA2R1 antigen” possesses at least the complete extracellular region of PLA2R1 or any variants thereof useful or sufficient to run such competition assays. In a preferred embodiment, the “extracellular region of PLA2R1”, as used herein with regard to any of the methods and other embodiments according to the present invention, is represented by SEQ ID NO1, and this is the sequence to which autoantibodies to be detected or the level of which is to be determined according to the present invention bind. In another preferred embodiment, a variant of SEQ ID NO1 may be used to practice the present invention, for example for designing methods and products according to the present invention. In particular, a polypeptide comprising SEQ ID NO1 or a variant thereof immobilized on a diagnostically useful carrier is provided or used. A polypeptide comprising the CysR-FnII-CTLD1 domain, preferably SEQ ID NO8, is a variant of the extracellular region PLA2R1 for certain embodiments of the invention.

The teachings of the present invention may not only be carried out using polypeptides having the wild-type sequences referred to in this application explicitly, such as SEQ ID NO: 1, for example by function, name, sequence or accession number, or implicitly, but variants thereof. In a preferred embodiment, the term “variant”, as used herein, refers to a polypeptide having at least 50, 55, 60, 65 ,70, 75, 80, 85, 90, 92, 94, 96, 97, 98, 99, 99.5 or 99.9% sequence identity with the full-length sequence referred to herein or at least a biologically active fragment thereof. The variant may be created by incorporating insertions, mutations, deletions into a wild-type sequence as well as fusing to the N- and/or C-terminus of the wild-type sequence or a variant thereof additional sequences, for example artificial linker and/or tag sequences. In a preferred embodiment, the variant comprises at least 30 successive amino acid residues from or derived from the wild-type sequence. Particularly preferred variants include homologous sequences from animals, preferably mammals.

The variant of the polypeptide has biological activity. In a preferred embodiment, such biological activity is the ability to bind to the antibody to be detected or the level of which is to be determined, from a sample from a patient. For example, a polypeptide comprising a variant of SEQ ID NO1 has the ability to bind to an autoantibody to a polypeptide comprising SEQ ID NO1 from a sample from a patient. In a more preferred embodiment, said autoantibody to a polypeptide comprising SEQ ID NO1 may comprise an autoantibody to CysR and an autoantibody to CTLD1. A variant useful for this is CysR-FnII-CTLD1. In another more preferred embodiment, said autoantibody to a polypeptide comprising SEQ ID NO1 may comprise an autoantibody to CysR, an autoantibody to CTLD1, an autoantibody to CTLD5, and autoantibody to CTLD7 and an autoantibody to CTLD8. The methods according to the present invention, for example the ELISA-based technology in the examples may be used to determine whether or not a variant has said biological activity.

Guidance for designing variants for the person skilled in the art is available in state-of-the-art documents disclosing which parts of the PLA2R1 sequence are important for the binding activity with regard to autoantibodies, for example in: 1) Fresquet M, Jowitt TA, Gummadova J, et al. Identification of a major epitope recognized by PLA2R autoantibodies in primary membranous nephropathy. J Am Soc Nephrol. 2015;26(2):302-313; 2) B. Seitz-Polski, G. Dolla, C. Payre, N.M. Tomas, M. Lochouam, L. Jeammet, C. Mariat, T. Krummel, S. Burtey, C. Courivaud, W. Schlumberger, K. Zorzi, S. Benzaken, G. Bernard, V.L. Esnault, G. Lambeau, Cross-reactivity of anti-PLA2R1 autoantibodies to rabbit and mouse PLA2R1 antigens and development of two novel ELISAs with different diagnostic performances in idiopathic membranous nephropathy, Biochimie, 118 (2015) 104-115; 3) Seitz-Polski, B, Dolla, G, Payre, C, Girard, CA, Polidori, J, Zorzi, K, Birgy-Barelli, E, Jullien, P, Courivaud, C, Krummel, T, Benzaken, S, Bernard, G, Burtey, S, Mariat, C, Esnault, VL, Lambeau, G: Epitope Spreading of Autoantibody Response to PLA2R Associates with Poor Prognosis in Membranous Nephropathy. J Am Soc Nephrol, 27: 1517-1533, 2016; 4) L. Reinhard, G. Zahner, S. Menzel, F. Koch-Nolte, R.A.K. Stahl, E. Hoxha, Clinical Relevance of Domain-Specific Phospholipase A2 Receptor 1 Antibody Levels in Patients with Membranous Nephropathy, J Am Soc Nephrol, 31 (2020) 197-207.

When designing variants, the person skilled in the art will bear in mind the purpose of the assay. In a preferred embodiment, a polypeptide comprising CTLD1 used to determine the level of autoantibodies to CTLD1 only will be designed such that it does not bind to autoantibodies to CysR. In another preferred embodiment, a polypeptide comprising CysR used to determine the level of autoantibodies to CysR only will be designed such that it does not bind to autoantibodies to CTLD1.

In a preferred embodiment, the antibody to be detected or the level of which is to be determined binds preferably specifically to the sequence of interest, such as the extracellular domain of PLA2R1, or the CysR, CTLD1, CTLD5, CTLD7 or CTLD8 domains. Specific binding preferably means that the binding reaction is stronger than a binding reaction characterized by a dissociation constant of 1 × 10⁻⁶ M, more preferably 1 × 10⁻⁷ M, more preferably 1 × 10⁻⁸ M, more preferably 1 × 10⁻⁹ M, more preferably 1 × 10⁻¹⁰ M, more preferably 1 × 10⁻¹¹ M, more preferably 1 × 10⁻¹² M, as determined for instance by surface plasmon resonance using a Biacore equipment or similar systems at 25° C. in a PBS buffer at pH 7.0.

Polypeptide competitors are selected among a CysR-FnII-CTLD1 fragment, a CysR fragment, a CTLD1 fragment, a CTLD5 fragment, a CTLD7 fragment, a mix of CysR and CTLD2-8 fragments (CysR+CTLD2-8), and a mix of CTLD1 and CTLD2-8 fragments (CTLD1+CTLD2-8) or a mixture thereof or any peptides thereof.

In an embodiment, the polypeptide competitors are chosen among a CysR fragment, a CTLD1 fragment, a CysR-FnII-CTLD1 fragment or a mixture thereof.

In another embodiment, the polypeptide competitors are chosen among a CysR fragment, a CTLD1 fragment, and CysR-FnII-CTLD1 fragment, a CTLD2-8 fragment or a mixture thereof.

In yet an embodiment, the polypeptide competitors are chosen among a CysR fragment, a CTLD1 fragment, a CysR-FnII-CTLD1 fragment, a CTLD7 fragment and a CTLD5 fragment or a mixture thereof.

In another embodiment, the polypeptide competitor is a CysR-FnII-CTLD1 fragment.

In another embodiment, the polypeptide competitors are a CysR fragment and a CTLD 1 fragment;

In a yet preferred embodiment, the polypeptide competitors are a CysR fragment, a CTLD1 fragment, and a CysR-FnII-CTLD1 fragment.

In a yet preferred embodiment, the polypeptide competitors are a CysR fragment, a CTLD1 fragment, a CysR-FnII-CTLD1 fragment, and a CTLD2-CTLD8 fragment.

In a yet preferred embodiment, the polypeptide competitors are a CysR fragment, a CTLD1 fragment, a CysR-FnII-CTLD1 fragment, a CTLD7 fragment, and a CTLD5 fragment.

Full PLA2R1 antigen and polypeptide competitors are recognized by autoantibodies.

The term “autoantibody” has its general meaning in the art and refers to an antibody that is produced by the immune system of a subject and that is directed against subject’s own proteins (for example specific epitopes in domains of PLA2R1). Autoantibodies may attack the body’s own cells, tissues, and/or organs, causing inflammation, cell injury and eventually tissue injury like podocyte injury and kidney injury in membranous nephropathy. In the present application the terms autoantibody or antibody are used in the same meaning.

As used herein, the expressions “autoantibodies directed against CysR-FnII-CTLD1, CysR, CTLD1, CTLD5, CTLD7, CTLD8, CysR+CTLD2-8, CTLD1+CTLD2-8”, “autoantibodies directed against polypeptide competitors” and “autoantibodies of the invention” refer to autoantibodies that respectively recognize the CysR-FnII-CTLD1 domain of PLA2R1, the cysteine-rich domain (CysR or CR) of PLA2R1; the C-type lectin-like domain 1 (CTLD1 or C1) of PLA2R1; the C-type lectin-like domain 5 (CTLD5 or C5) of PLA2R1, the C-type lectin-like domain 7 (CTLD7 or C7) of PLA2R1, the C-type lectin-like domain 8 (CTLD8 or C8) of PLA2R1, a mix of CysR and CTLD2-8 domains of PLA2R1, and a mix of CTLD1 and CTLD2-8 domains of PLA2R1.

The method according to the invention comprises a step of determining the nature of the antibody which mostly drives the humoral response in a sample by performing a competition assay, said competition assay comprising the steps of:
first incubating a sample from a patient suffering from membranous nephropathy with saturating amounts of a CysR-FnII-CTLD1 fragment,
contacting the sample with the full PLA2R1 antigen;
measuring the remaining activity.

In a preferred embodiment, the competition assay comprises the step of:
first incubating a sample from a patient suffering from membranous nephropathy with saturating amounts of polypeptide competitors selected among a CysR fragment, a CTLD1 fragment, a CysR-FnII-CTLD1 fragment or a mixture thereof;
contacting the sample with the full PLA2R1 antigen;
measuring the remaining activity.

In a yet preferred embodiment, the competition assay comprises the step of:
first incubating a sample from a patient suffering from membranous nephropathy with saturating amounts of polypeptide competitors selected among a CysR fragment, a CTLD1 fragment, a CysR-FnII-CTLD1 fragment, a CTLD2-8 fragment or a mixture thereof;
contacting the sample with the full PLA2R1 antigen;
measuring the remaining activity.

In a yet preferred embodiment, the competition assay comprises the step of:
first incubating a sample from a patient suffering from membranous nephropathy with saturating amounts of polypeptide competitors selected among a CysR fragment, a CTLD1 fragment, a CysR-FnII-CTLD1 fragment, a CTLD5 fragment and a CTLD7 fragment or a mixture thereof;
contacting the sample with the full PLA2R1 antigen;
measuring the remaining activity.

As used herein, the terms “remaining activity” refers to the percentage of remaining signal measured in the presence of at least one polypeptide competitor, taking into account the non-specific signal for individual patient. This percentage is compared to the maximal specific activity (100%) measured in the absence of any polypeptide competitor. The respective remaining activity measured in the presence of the various PLA2R1 domains or fragments allows to determine the type of immunodominance.

In a preferred embodiment, the competition assay is performed by ELISA. The term “ELISA” as used herein means an enzyme linked immunosorbent assay. Competitive ELISA is a type of competitive binding assay comprising antibodies and a detectable label used to quantitate the amount of an analyte in a sample.

An ELISA format of such a competition assay is a preferred format, but any type of immunocompetition assay may be conducted, including but not limited to cell-based assays, multi-array plates, immunoprecipitations, immunodepletions, dot blots or western blots.

The method of the invention thus allows measuring the remaining activity wherein the sample obtained from the patient is first pre-incubated with an excess of polypeptide competitors and then tested against full PLA2R1 antigen.

Typically, in the context of the ELISA competition assay, microplates are coated with the purified full extracellular domain of PLA2R1. The biological samples are preincubated with saturating amounts of different recombinant proteins (one or several domains of PLA2R1 or variants thereof) and then added to wells coated with full PLA2R1 antigen and incubated. The plate can be washed to remove unbound moieties and a detectably labelled secondary binding antibody or any other secondary binding molecule is added. The secondary binding molecule is allowed to react with any captured human autoantibody, the plate is washed and the presence of the secondary binding antibody or molecule is detected using methods well known in the art.

In other preferred embodiments, other assays can be used according to any embodiment of the present invention such as a competition assay, in particular from the group comprising immunodiffusion assays, immunoelectrophoretic assays, light scattering assays, agglutination assays, labeled immunoassays such as those from the group comprising radiolabeled assays, enzyme assays such as colorimetric assays, chemiluminescence assays and immunofluorescence assays.

In a preferred embodiment, to perform an assay according to any embodiment of the present invention, a polypeptide selected from the group comprising CysR, CTLD1, PLA2R1, the extracellular domain of PLA2R1, CTLD5, CTLD7 and CTLD8 may be immobilized on a diagnostically useful carrier, preferably from the group comprising glass slide, preferably for microscopy, a biochip, a microarray, a microtiter plate, a lateral flow device, a test strip, a membrane, e.g. a nitrocellulose membrane, preferably a line blot, a chromatography column and a bead, preferably a microtiter plate. Such carrier may be in a kit. Both carrier and kit may be used for the purpose of any method according to the present invention.

It is noteworthy that human anti-PLA2R1 autoantibodies are selected among the following isotypes: IgG1, IgG2, IgG3 and IgG4. Preferably, human anti-PLA2R1 autoantibody is an IgG4. Thus, detection with labelled secondary binding antibodies include detection for any of these IgG subclasses including detection using anti-total IgG or any other detection systems such as protein A or G, with any labelling well known in the art.

Basically, the polypeptide competitors used for carrying out the ELISA in the context of the invention are produced in HEK293 cells or any other protein expression systems such as but not limited to other mammalian cells, insect cells, yeast, plants or E. coli, or in vitro translation systems.

The full extracellular domain of PLA2R1 is typically expressed as a recombinant secreted protein from HEK293 cells, but can be produced by other means such as other protein expression systems including the use of other mammalian or non-mammalian host cells.

Results of competition assays are expressed as the percentage of maximal signal measured in the absence of competitor, taking into account the non-specific signal for individual patient.

### Measurement of the Remaining Activity, Immunodominance Profiling and Patients’ Stratification Based on Immunodominance Profiling

For the majority of patients, the N-terminal CysR-FnII-CTLD1 triple domain could inhibit from 50 to 100% of the PLA2R1 signal, preferably from 60 to 100% of the PLA2R1 signal and more preferably from 65 to 100% of the PLA2R1 signal, indicating that the autoimmune response is mostly driven by autoantibodies targeting CysR and/or CTLD1 epitope-containing domains.

Further competition assays with CysR or CTLD1 as single domains as well as with CysR or CTLD1 mixed with CTLD2-8 demonstrated that both CysR and CTLD1 are major contributors of anti-PLA2R1 reactivity. In contrast, further competition assays with the C-terminal CTLD5 and CTLD7 epitope-containing domains showed that these latter domains are minor contributors of anti-PLA2R1 reactivity for most patients.

Hence, the method according to the invention allows determining the nature of the antibody which mostly drives the humoral response.

Based on these results, patients can be stratified into three groups: immunodominant CysR (iCR), immunodominant CTLD1 (iC1) and non-immunodominant (non-iDom).

When competition with a CysR-FnII-CTLD1 fragment is higher than 50% while competition with a CTLD1 fragment is lower than 30%, and preferably when competition with a CysR-FnII-CTLD1 fragment is higher than 60% while competition with a CTLD1 fragment is lower than 25%, and yet preferably, when competition with a CysR-FnII-CTLD1 fragment is higher than 65% while competition with a CTLD1 fragment is lower than 20%, then the humoral response is mostly driven by anti-CysR autoantibodies. This defines patients who are immunodominant for CysR (iCR). iCR patients are hence defined by a humoral response mostly driven by autoantibodies targeting CysR and a low contribution of autoantibodies recognizing other epitope containing domains including CTLD1. The patient is “immunodominant for CysR”.

When competition with a CysR-FnII-CTLD1 fragment is higher than 50% while competition with a CTLD1 fragment is higher than 10%, and preferably when competition with a CysR-FnII-CTLD1 fragment is higher than 60% while competition with a CTLD1 fragment is higher than 15% and preferably, when competition with a CysR-FnII-CTLD1 fragment is higher than 65% while competition with a CTLD1 fragment is higher than 20%, then the humoral response is driven by anti-CTLD1 autoantibodies, yet with the respective contribution of anti-CysR autoantibodies. This defines patients who are immunodominant for CTLD1 (iC1). iC1 patients are thus defined by a humoral response driven not only by anti-CysR but also by anti-CTLD1 autoantibodies, with these latter contributing up to 80% of the PLA2R1 signal reactivity (i.e. with a balanced and gradual increase of anti-CTLD1 reactivity at the expense of anti-CysR reactivity) and little contribution from other distal epitope containing domains. The patient is “immunodominant for CTLD1”.

When competition with a CysR-FnII-CTLD1 fragment is lower than 50%, preferably lower than 60% and yet preferably lower than 65% while competition with any individual domain is too low to determine any specific type of immunodominance, meaning that no specific domain was driving the signal of the humoral autoimmune response, the PLA2R1 signal appears uniformly spread over the different epitope-containing domains without indication of immunodominance by a particular epitope domain. This defines patients who are non-immunodominant (non-iDom). Non-iDom patients are hence defined by a humoral response where the PLA2R1 signal appears to be uniformly spread over the different epitope-containing domains without any indication of immunodominance by a particular specific epitope-containing domain. The patient is “non-immunodominant”.

The inventors clearly showed that the five PLA2R1 domains containing independent epitopes are not equivalent in terms of immunological reactivity. Among them, the N-terminal CysR domain plays a central role and is undoubtedly the major immunodominant epitope-containing domain, based on both its highest prevalence (100%) and its highest contribution to the anti-PLA2R1 signal as measured by competition ELISA (up to 100%) among patients. However, while all patients were positive for CysR, not all of them were iCR, and they can be iC1 or non-iDom. iCR patients were distributed among the three tertiles of anti-PLA2R1 titer, yet more present in the first and second tertiles. In a preferred embodiment, “CysR”, as used herein with regard to any of the methods and other embodiments according to the present invention, is represented by SEQ ID NO2, and this is the sequence to which autoantibodies to be detected or the level of which is to be determined according to the present invention bind. In another preferred embodiment, a variant of SEQ ID NO2 may be used to practice the present invention, for example for designing methods and products according to the present invention. In particular, a polypeptide comprising SEQ ID NO2 or a variant thereof immobilized on a diagnostically useful carrier is provided or used.

The CTLD1 domain was clearly identified as a second immunodominant epitope-containing domain with specific features. The inventors indeed demonstrated that CysR and CTLD1 domains contain independent epitopes, while the FnII domain does not harbor any. In contrast to CysR, the prevalence for CTLD1 positivity was only 46.5%, with positivity more often observed in the second and third tertiles of anti-PLA2R1 titer. Also in contrast to CysR, iC1 patients were more often present in the high tertile with an opposite trend for iCR. Together, this suggests a switch from iCR to iC1 among patients as the anti-PLA2R1 titer increases. In a preferred embodiment, “CTLD1”, as used herein with regard to any of the methods and other embodiments according to the present invention, is represented by SEQ ID NO3, and this is the sequence to which autoantibodies to be detected or the level of which is to be determined according to the present invention bind. In another preferred embodiment, a variant of SEQ ID NO3 may be used to practice the present invention, for example for designing methods and products according to the present invention. In particular, a polypeptide comprising SEQ ID NO3 or a variant thereof immobilized on a diagnostically useful carrier is provided or used.

CTLD5 was identified as a new independent and highly prevalent but not immunodominant epitope-containing domain, with features clearly different from CTLD1 and CysR. First, CTLD5 was the second most prevalent epitope-containing domain with 65.5% positivity, irrespective of positivity for CTLD1 or other domains. Accordingly, patients with a CTLD5 profile were most prevalent. Second, like CysR but unlike CTLD1, CTLD5 positivity was spread all over the three anti-PLA2R1 tertiles, suggesting that patients may become positive to CTLD5 at early steps during the maturation of the autoimmune response. Third, in contrast to both CysR and CTLD1, CTLD5 had a very minor contribution to the anti-PLA2R1 titer as measured by competition ELISA. Finally, CTLD5 differs from other epitope-containing domains by its particular reactivity to patients’ autoantibodies, especially by western blot versus ELISA, but also other techniques of immunology. In a preferred embodiment, the extracellular region of “CTLD5”, as used herein with regard to any of the methods and other embodiments according to the present invention, is represented by SEQ ID NO4, and this is the sequence to which autoantibodies to be detected or the level of which is to be determined according to the present invention bind. In another preferred embodiment, a variant of SEQ ID NO4 may be used to practice the present invention, for example for designing methods and products according to the present invention. In particular, a polypeptide comprising SEQ ID NO4 or a variant thereof immobilized on a diagnostically useful carrier is provided or used.

CTLD7, previously identified as an epitope-containing domain was confirmed as a fourth independent epitope-containing domain. Its prevalence and pattern of positivity was comparable to CTLD1, with positivity associated with high titer. However, in contrast to CTLD1, the contribution of CTLD7 to the anti-PLA2R1 titer was modest, and positivity for CTLD7 was independent of positivity for CTLD1 or immunodominance towards the iCR or iC1 pathways. In a preferred embodiment, “CTLD7”, as used herein with regard to any of the methods and other embodiments according to the present invention, is represented by SEQ ID NO5, and this is the sequence to which autoantibodies to be detected or the level of which is to be determined according to the present invention bind. In another preferred embodiment, a variant of SEQ ID NO5 may be used to practice the present invention, for example for designing methods and products according to the present invention. In particular, a polypeptide comprising SEQ ID NO5 or a variant thereof immobilized on a diagnostically useful carrier is provided or used.

Last, CTLD8 was identified as the most C-terminal and minor epitope-containing domain. Like CTLD1 and CTLD7, positivity for CTLD8 was associated with high titer, with CTLD8 contributing very little to the anti-PLA2R1 titer. In a preferred embodiment, the “CTLD8”, as used herein with regard to any of the methods and other embodiments according to the present invention, is represented by SEQ ID NO6, and this is the sequence to which autoantibodies to be detected or the level of which is to be determined according to the present invention bind. In another preferred embodiment, a variant of SEQ ID NO6 may be used to practice the present invention, for example for designing methods and products according to the present invention. In particular, a polypeptide comprising SEQ ID NO6 or a variant thereof immobilized on a diagnostically useful carrier is provided or used.

Without wishing to be bound by theory, the inventors inferred that the most mature humoral autoimmune response with the highest anti-PLA2R1 titers may be reminiscent of a mechanism of epitope spreading associated with immunodominance toward specific epitopes. This mechanism may start before the overt phase of the disease, i.e. during the smoldering phase of MN, with progression over months or even years from the early onset of the autoimmune response to the clinical signs of the disease; and may still progress during overt disease, with fluctuation of the autoimmune response over phases of remission and relapse. Within this mechanism of epitope spreading, it is tempting to speculate that the autoimmune response would have been initiated on the CysR domain and would have then matured by intramolecular epitope spreading up to the C-terminal CTLD8 domain, with possible concomitant 1) intradomain epitope spreading within the CysR and/or CTLD1 domains becoming the two immunodominant domains and 2) interdomain epitope spreading towards the CTLD5, CTLD7 and CTLD8 domains harboring only non-immunodominant epitopes. Of note, all the reactivity to these domains appears to be due to conformational epitopes, with IgG4 as the predominant IgG subclass for all epitopes.

The anti-PLA2R1 titer increases as the number of positive epitopes also increases, and the highest titers can only be observed when either CysR or CTLD1 or both play an immunodominant role and maximally drive the humoral autoimmune response. The non-iDom group of patients would correspond to the rare cases where neither CysR nor CTLD1 are immunodominant (but may eventually become during patients’ follow-up), explaining the relatively low anti-PLA2R1 titers measured in those patients.

These data suggest a switch of immunodominance from CysR to CTLD1 once this latter becomes positive, associated with a maximal autoimmune reaction for epitope spreading and titer, and worsening of disease activity. In addition, the respective immunodominance towards the iCR or iC1 pathways may be due to genetic factors, since PLA2R1 gene polymorphisms on both CysR and CTLD1 domains have been associated with predisposition to develop PLA2R1-associated MN.

Importantly, all patients had anti-CysR autoantibodies but only about half of them (46.5%) had anti-CTLD1 autoantibodies. Furthermore, 55% of all patients were iCR and 36% were iC1. This implies an “imbalance” between the two groups which may be important at the pathophysiological level. For the major group of iCR patients, the autoimmune response is mostly driven by anti-CysR autoantibodies acting as a “single class” of autoantibodies and targeting PLA2R1 at a single binding domain that is CysR. In contrast, for iC1 patients, the autoimmune response is driven by different ratios of anti-CysR and anti-CTLD1 autoantibodies, acting as a “dual class” of autoantibodies and targeting PLA2R1 at two different binding domains, which might lead to larger immune deposits, more podocyte injury and an overall increased pathogenicity.

### Analysis of Anti-CTLD1/Anti-CysR Ratio as a Surrogate of Competition ELISA To Determine Immunodominance

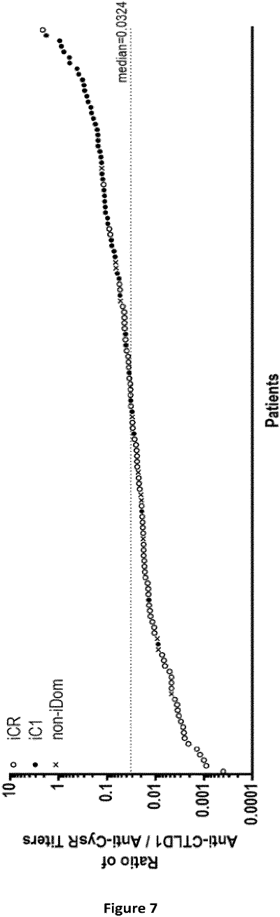

The authors also show that measurement of the ratio between anti-CTLD1 and anti-CysR antibody titers in a sample obtained from a patient suffering from membranous nephropathy can be used as an alternative method to determine the immunodominant profile of the patient (see FIGS. 7-9 and the results below).

Anti-CTLD1 and anti-CysR titers can be measured as mentioned above and the ratio of anti-CTLD1/anti-CysR titers (or alternatively anti-CysR/anti-CTLD1 antibody titers) is calculated.

By “by establishing a ratio between the titers of the anti-CTLD1 antibodies and the anti-CysR antibodies”, it is herein intended without further precision, that the ratio can be established either as anti-CTLD1 antibody titer/anti-CysR antibody titer or as anti-CysR antibody titer/anti-CTLD 1 antibody titer.

As shown in FIG. 7, most patients with a ratio below the median value are iCR while most patients above the median are iC1, indicating a good concordance between the methods as described herein to assess immunodominance (i.e., competition ELISA and analysis of the ratio).

The ratio value can be compared to a reference value. Typically, when the ratio of anti-CTLD1 antibody titer above anti-CysR antibody titer is performed, it can be considered that the patient is immunodominant for CTLD1 (iC1) when said ratio is above a given reference value. If the ratio is below the given reference value, then the patient is considered not immunodominant for CTLD1 and immunodominant for CysR. Alternatively, when the ratio of anti-CysR antibody titer above anti-CTLD1 antibody titer is performed, it can be considered that the patient is immunodominant for CTLD1 (iC1) when said ratio is below a given reference value. If the ratio is above the given reference value, then the patient is considered not immunodominant for CTLD 1 and immunodominant for CysR.

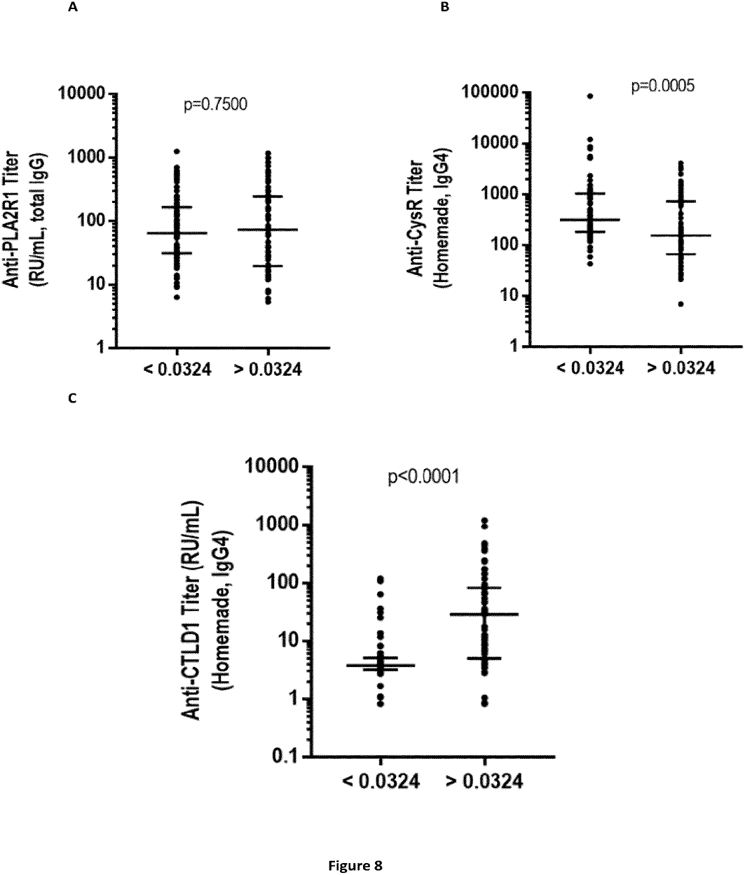

Typically, a reference value can be established from a reference population of subjects suffering from membranous nephropathy, typically as illustrated in FIG. 7 (see also the corresponding result section). Typically, the median ratio value for the population can be selected as a reference value. For example in the selected population, the median ratio value is 0.0324 (see FIG. 7) As illustrated, patients with a ratio below the median can be considered as immunodominant for CysR (iCR) while patients with a ratio above the median can be considered as immunodominant for CTLD1 (iC1). Furthermore, as expected from the two immunodominant pathways of immunodominance towards either CysR or CTLD1 epitope-containing domains, patients with a ratio of anti-CTLD1/anti-CysR titers below or above the median have similar anti-PLA2R1 titers, indicating that immunodominance is independent of the full titer, as determined by the standardized ELISA (FIG. 8). In some embodiments, the reference value can also be a fixed reference value.

### Method of Predicting the Prognosis of a Patient Suffering From Membranous Nephropathy

Multiple studies in the state of the art, have demonstrated the clinical value of measuring anti-PLA2R1 titer to better predict clinical outcome, with low titer associated with a higher chance to reach remission, either spontaneous or after immunosuppressive treatment, and high titer associated with an increased risk of progression to severe disease and end-stage kidney disease (ESKD), as well as resistance to treatment (Ruggenenti, P, Fervenza, FC, Remuzzi, G: Treatment of membranous nephropathy: time for a paradigm shift. Nat Rev Nephrol, 13: 563-579, 2017).

The inventors previously demonstrated that profiling of patients based on epitope positivity towards a single (CysR) or multiple (CysR and CTLD1 and/or CTLD7) epitope-containing domains may also help to better predict clinical outcome and guide efficient therapy (WO2017/009245). Indeed, it has been demonstrated that the presence of autoantibodies directed against both CysR and CTLD1 and/or CTLD7 domains of PLA2R1 are relevant for establishing a poor prognosis of the disease. On the contrary, a patient presenting autoantibodies directed only against the CysR domain of PLA2R1 has a good prognosis.

In the present invention, a retrospective but well-characterized cohort of patients with PLA2R1-associated MN has been established in which about one half of the patients were treated with conservative therapy (NIAT, 43%) and the other half with the immunosuppressant rituximab (43%).

The inventors tested side-by-side whether anti-PLA2R1 titer and immunodominance can be used as predictors of clinical outcome.

The inventors demonstrate that the consideration of immunodominance defining three groups (iCR, non-iDom and iC1) or two groups (iCR/non-iDom versus iC1) of patients showed that immunodominance can predict clinical outcome.

The present invention and its associated methods (competition assays and analysis of titer ratio) is more accurate and precise compared to the state of the art and can advantageously avoid false positive and/or negative.

The inventors demonstrate that immunodominance can be assessed by competition assays or through analysis of the ratio of measured anti-CTLD1 vs. anti-CysR titers. The results included herein provide evidence that assessment of immunodominance (either by competition assay or by analysis of CTLD1 vs CysR – or conversely – antibody titer ratio) is more accurate to predict clinical outcome.

Hence, in a second aspect, the present invention relates to a method of predicting the prognosis of a patient suffering from membranous nephropathy comprising determining PLA2R1 immunodominance in a sample obtained from said patient according to the method described above, wherein:
a patient immunodominant for CTLD1 exhibits a poor prognosis;
a patient non-immunodominant for CTLD1 (typically immunodominant for CysR and/or a non-immunodominant - if immunodominance is assessed by competition assay) patient exhibits a good prognosis.

The inventors indeed identified that patients reaching remission were more often iCR (and/or non-iDom) than iC1, demonstrating that immunodominance is a novel predicting factor of clinical outcome. It has been demonstrated that iC1 patients had about 3-fold lower chance to reach remission than iCR (and/or non-iDom patients).

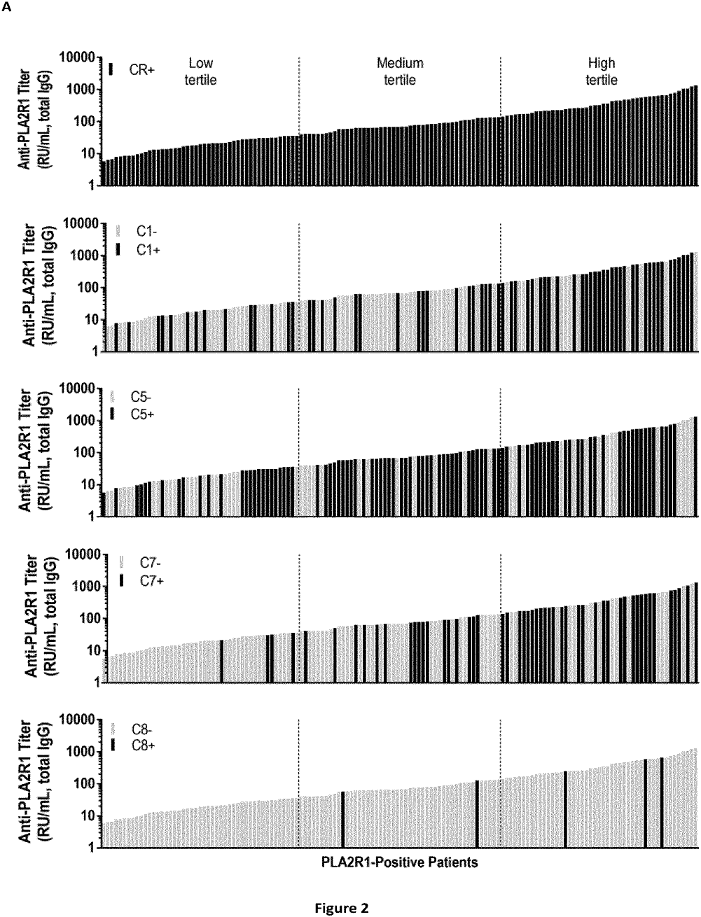

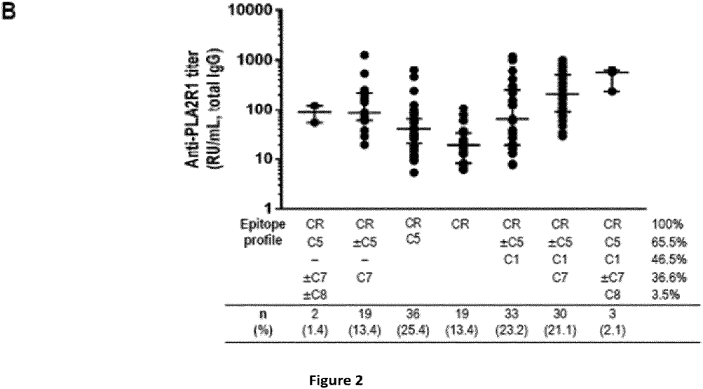

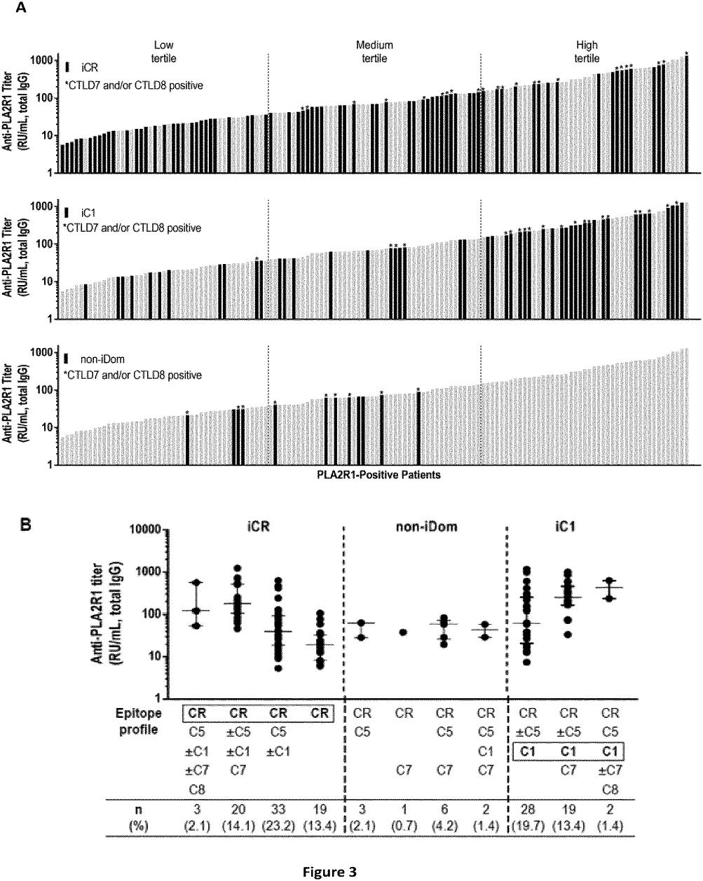

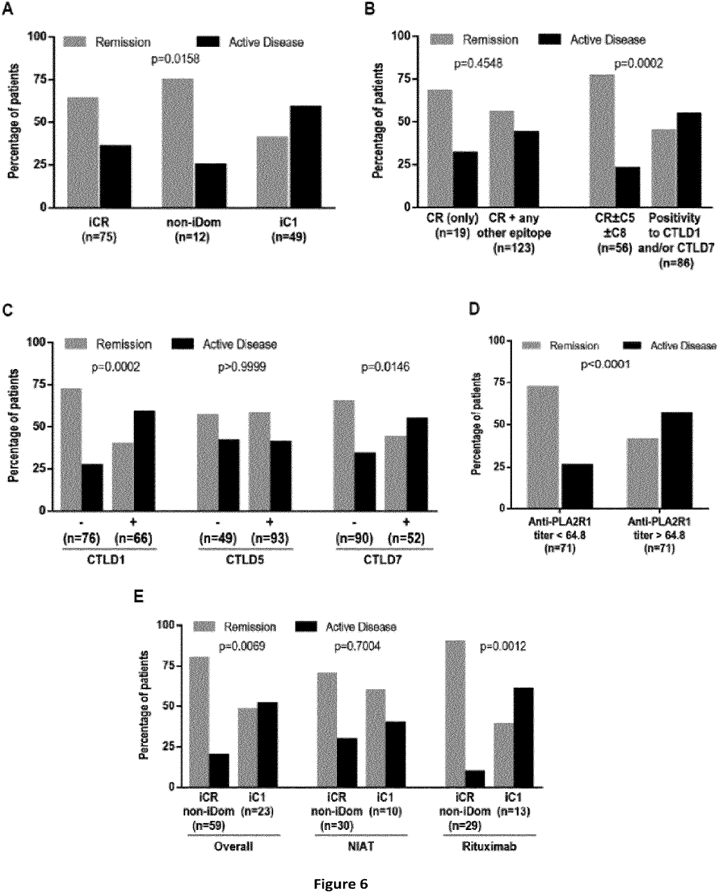

The method according to the invention, based on immunodominance determination (by competition assay or by measuring the ratio of anti-CTLD1 antibody titer and anti-CysR antibody titer - or conversely) allows to more accurately stratify patients, compared to the methods described in the state of the art, typically full anti-PLA2R1 titer and profiling by epitope positivity (see FIGS. 6-9). The relationships between titer, epitope profile and immunodominance towards the iCR and iC1 pathways are illustrated in FIGS. 2 and 3. Determination of the immunodominant profile according to the present invention allows distinguishing these patients differentially and more accurately.

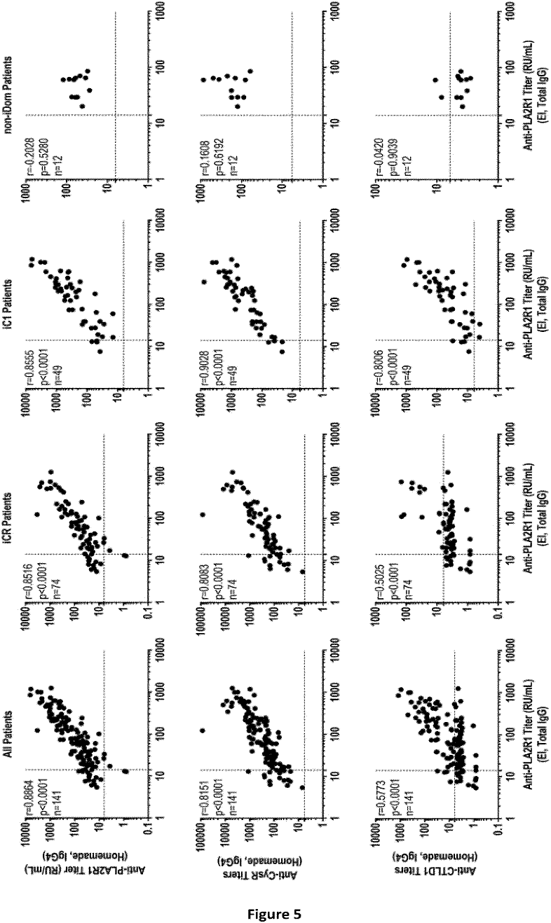

In our study cohort, anti-PLA2R1 titer in iC1 patients who exhibit a poor prognosis varies from 7.5 to 1,183 RU/mL, as measured by the standardized ELISA (FIG. 5). It is to be noted that anti-PLA2R1 titer can be typically measured using the standardized and commercially available Euroimmun (Medizinische Labordiagnostika AG, Lübeck, Germany) standard tests, as also detailed in Dahnrich C, Komorowski L, Probst C, Seitz-Polski B, Esnault V, Wetzels JF, Hofstra JM, Hoxha E, Stahl RA, Lambeau G, et al. “Development of a standardized ELISA for the determination of autoantibodies against human M-type phospholipase A2 receptor in primary membranous nephropathy. Clin Chim Acta. 2013;421C(213-8)”. In addition, anti-CTLD1 titer varies between about 10 to 1,500 RU/mL (in-house ELISA, FIG. 5), which means that neither anti-PLA2R1 titer nor anti-CTLD1 titer alone is sufficient to accurately categorize patients. Indeed, patients having a low anti-PLA2R1 titer can exhibit a poor prognosis, a good prognosis or an intermediate prognosis. Moreover, non-iDom and iCR patients have similar median anti-PLA2R1 titer (respectively 59.7 and 56.5 RU/mL). Anti-PLA2R1 titer would not allow to accurately distinguish among those patients which ones have a good, poor, better or worse clinical outcome, especially among patients within a narrow range of low, medium or high titers but identified with variable clinical outcomes or response to treatment (see below and FIG. 6).

Furthermore, in the state of the art, it has been demonstrated that patients with autoantibodies directed against CTLD1 and/or CTLD7 domains (also referred to as epitope profile or epitope spreading) have a poor prognosis. On the contrary, patients with autoantibodies only directed against the CysR domain have a good prognosis.

Hence, when considering epitope profile or epitope spreading, a patient presenting autoantibodies directed only against the CysR domain of PLA2R1 (also called “non-spreader” patient) has a good prognosis, and a patient presenting autoantibodies directed against the CTLD 1 and/or CTLD7 domains of PLA2R1 (also called “spreader” patient) has a poor prognosis.

This stratification based on epitope profile or spreading may however lead to false-positive and false-negative cases in predicting clinical outcome and response to treatment.

With the present method based on immunodominance profiling, the inventors demonstrate that patients having anti-CTLD1 autoantibodies but not classified as immunodominant for CTLD1 (i.e. iC1), which would have been previously characterized as spreaders with poor prognosis, can now be classified as iCR (and/or non-iDom when immunodominance is assessed by competition assay) patients with a good prognosis.

Similarly, patients with anti-PLA2R1 antibodies beyond anti-CysR, including for instance anti-CTLD7, would have been classified as spreaders with poor prognosis, but can now be classified as iCR (and/or optionally non-iDom if immunodominance is assessed by competition assay) having a better prognosis than spreaders or iC1 patients.

Hence the new method of stratification according to the invention does not correspond to a mere determination of PLA2R1 immunodominant profile but advantageously allows classifying more accurately patients suffering from membranous nephropathy and avoiding false-positive (patients having anti-CTLD1 autoantibodies or autoantibodies other than anti-CysR) and false-negative (patients exhibiting only anti-CysR autoantibodies).

The expression “prognosis” as used herein refers to predicting the course or outcome of membranous nephropathy, preferably PLA2R1-associated nephropathy condition in a subject. This does not refer to the ability to predict the course or outcome of a condition with 100% accuracy, or even that a given course or outcome is predictably more or less likely to occur based on the pattern of biomarkers. Instead, the person skilled in the art will understand that the expression “prognosis” refers to an increased probability that a certain course or outcome will occur.

In the context of the present invention, “good prognosis” means a better prognosis, and refers to a higher chance of remission, either spontaneous or induced by treatment with immunosuppressants, and/or preferably a lower risk of requiring hemodialysis and/or a lower risk of developing kidney failure.

Patients considered as having a good prognosis according to the method of the invention would thus not be in need of hemodialysis, if appropriately treated. Further, said subjects would not need to be subjected to a more aggressive immunosuppressive treatment. Typically, “spontaneous remission” is defined by remission induced by symptomatic treatment (such as the use of RAS blockers and diuretics, also referred to as NIAT treatment) without immunosuppressive treatment.

In the context of the present invention, “poor prognosis” refers to a higher chance of onset of subsequent renal complication, such a sustained active MN disease possibly leading to end-stage kidney failure (ESKD).

Poor prognosis is typically associated with:
an increased proteinuria, typically a proteinuria > 3.5 g/g; and/or
a serum creatinine increased over 30%; and/or
an estimated glomerular filtration rate (eGFR) < 45 mL/min/1.73 m².
The eGFR is used to screen for and detect early kidney damage and to monitor kidney status. It is performed by doing a creatinine test and calculating the estimated glomerular filtration rate.

Typically, subjects considered as having a poor prognosis according to the method of the invention may need repeated treatments with effective doses of first-line immunosuppressants such as rituximab or would need alternative or combined therapies with stronger and more effective immunosuppressants such as cyclophosphamide, or would need hemodialysis.

### Method of Predicting the Prognosis of a Patient Suffering From Membranous Nephropathy and Resistance to Immunosuppressive Treatment

When combining immunodominance and treatment, iCR (and/or optionally non-iDom patients in the case where immunodominance is assessed by a competition assay) treated with immunosuppressants such as rituximab had about 3-fold more chance to reach remission than NIAT-treated patients. Furthermore, iCR (and/or non-iDom) patients treated with immunosuppressants such as rituximab had 4.5-fold more chance to enter into remission than iC1 patients also treated with immunosuppressants such as rituximab.

Hence, the present invention relates, in a third aspect, to a method of predicting the response to an immunosuppressant such as rituximab of a patient suffering from membranous nephropathy comprising determining PLA2R1 immunodominance in a sample obtained from said patient according to the method previously described, wherein:
a patient immunodominant for CTLD1 (iC1 patient) is resistant to immunosuppressant;
a patient non-immunodominant for CTLD1 (i.e. immunodominant for CysR or optionally non-immunodominant when immunodominance is assessed by a competition assay) is a good responder to immunosuppressant;

Current treatment for membranous nephropathy includes symptomatic conservative therapy based on RAS blockers and diuretics and/or immunosuppressive therapy with various types of immunosuppressants, among which rituximab appears as the preferred first-line therapy, based on efficiency versus secondary side effects.

Typically, about two thirds of patients will progress to severe membranous nephropathy and will require immunosuppressive therapy. Said immunosuppressive therapy is typically based on the administration of at least one compound selected but not limited from a group consisting of cyclosporin, tacrolimus, azathioprine, infliximab, omalizumab, daclizumab, adalimumab, eculizumab, efalizumab, natalizumab, omalizumab, rapamycin, cyclophosphamide, chlorambucil, rituximab, daratumumab, isatuximab and bortezomib.

Preferably, the treatment of idiopathic membranous nephropathy is based on the use of rituximab, cyclophosphamide, chlorambucil, tacrolimus.

Symptomatic treatment is typically based on blockade with RAS blockers (inhibitors of the renin-angiotensin system) and diuretics.

The invention also provides a mean by which a practitioner may predict the response of a patient subjected to a treatment, especially to immunosuppressants such as rituximab.

In the context of the invention, a patient is considered “resistant to treatment” when the patient is more resistant to treatment with immunosuppressants such as rituximab, shown by no amelioration or deterioration of the clinical parameters during follow-up and after administration of the immunosuppressant.

In the context of the invention, a patient is considered as a “good responder to treatment”, when the patient respond or is a better responder to treatment with immunosuppressants such as rituximab, shown by amelioration of the clinical parameters during follow-up and after administration of the immunosuppressant. It could refer to a lower risk of developing kidney failure and/or a lower risk of requiring hemodialysis. It could also refer to a patient who fully or partially restores clinical parameters to normal range including proteinuria and/or serum creatinine and/or estimated glomerular filtration rate (eGFR).

Typically, when a patient is considered resistant to immunosuppressant, the immunosuppressant has to be modified. Typically higher doses of immunosuppressant or repeated treatments, or alternative or combined therapy or a more aggressive immunosuppressant can be administered or a hemodialysis has to be initiated.

Typically, when a patient is considered as a good responder to immunosuppressant, the immunosuppressant has to be maintained until remission (complete or partial remission) or a symptomatic treatment can be administered.

In a preferred embodiment, the immunosuppressant is rituximab.

In another preferred embodiment, the method of predicting the response to an immunosuppressant of a patient suffering from membranous nephropathy comprises a further step of determining the anti-PLA2R1 titer.

Hence, in another embodiment, the method of predicting the response to an immunosuppressant of a patient suffering from membranous nephropathy comprises a further step of measuring the level of autoantibodies directed against PLA2R1 in the biological sample.

Methods for measuring the levels of autoantibodies in a biological sample may be measured by using standard immunodiagnostic techniques, including immunoassays such as competition, direct reaction, or sandwich-type assays. Such assays include, but are not limited to, agglutination tests; enzyme-labeled and -mediated immunoassays such as ELISAs; biotin/avidin type assays; radioimmunoassays; immunoelectrophoresis; immunoprecipitation. The reactions generally include revealing labels such as fluorescent, chemiluminescent, radioactive, enzymatic labels or dye molecules, or other methods for detecting the formation of a complex between the antigen and the antibody or antibodies reacted therewith.

In a preferred embodiment, the step of measuring the level of autoantibodies directed against PLA2R1 is performed by ELISA.

In a preferred embodiment, the anti-PLA2R1 titer is measured by the standardized ELISA (Euroimmun Medizinische Labordiagnostika AG, Lübeck, Germany, as also detailed in Dahnrich C, Komorowski L, Probst C, Seitz-Polski B, Esnault V, Wetzels JF, Hofstra JM, Hoxha E, Stahl RA, Lambeau G, et al. “Development of a standardized ELISA for the determination of autoantibodies against human M-type phospholipase A2 receptor in primary membranous nephropathy. Clin Chim Acta. 2013;421C(213-8)”), and is lower than 300 RU/mL, preferably lower than 250 RU/mL, preferably lower than 225 RU/mL, lower than 200 RU/mL, lower that 150 RU/mL or lower than 100 RU/mL.

In a yet preferred embodiment, the anti-PLA2R1 titer is lower than 200 RU/mL.

Indeed, the inventors highlight that patients best responding to rituximab are those with anti-PLA2R1 titers lower than 200 RU/mL.

The combined evaluation of anti-PLA2R1 titer (i.e. by selecting patients with a titer lower than 200 RU/mL) and immunodominance help to refine the likelihood of response to treatment, with immunodominance identifying patients best responding to rituximab (iCR/non-iDom, good responders to treatment) versus those poorly responding (iC1 patients, resistant to treatment).

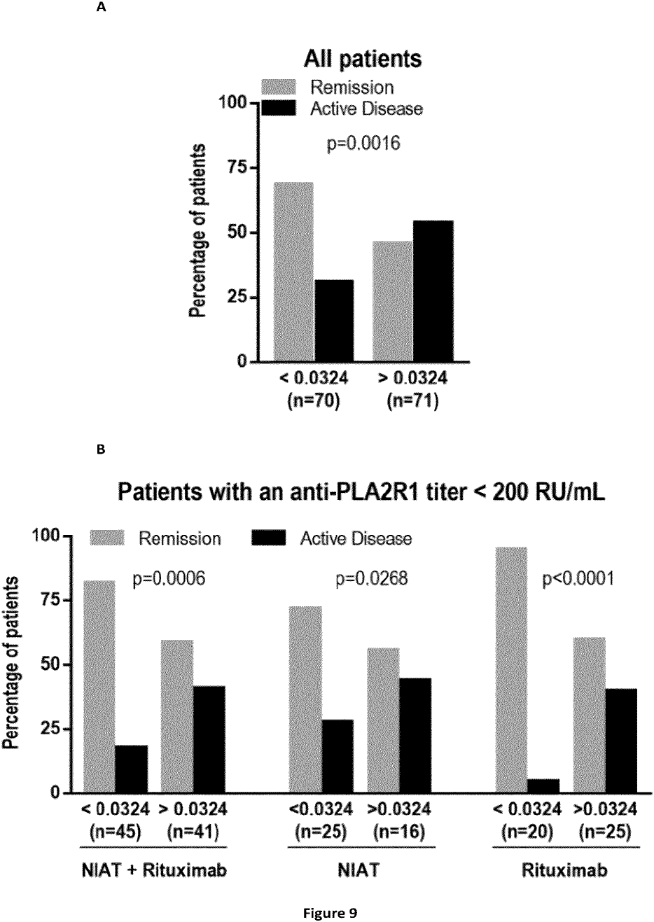

Specifically, below 200 RU/mL, the percentage of clinical remission in iCR (and/or optionally non-iDom patients) was higher than in iC1 patients, either when considering the overall clinical outcome or the one after rituximab treatment (FIG. 6E). When immunodominance is assessed by antibody titer ratio, the inventors also demonstrated that patients with a ratio of anti-CTLD1/anti-CysR below the median were more often in clinical remission than patients with a ratio above the median, when considering the overall clinical outcome or the one after rituximab treatment (FIG. 9B).

This suggests that below a certain cut-off for anti-PLA2R1 titer, immunodominance is critically useful to guide therapy and identify patients as responders versus non-responders to rituximab.

Similarly, patients with a ratio of anti-CTLD1/anti-CysR below the median were more often in clinical remission than patients with a ratio above the median, when considering the overall clinical outcome or the one after rituximab treatment. Typically, when the patient is considered resistant to rituximab (iC1 patients), and the anti-PLA2R1 titer is lower than 300 RU/mL, notably lower that 250 RU/mL, lower that 200 RU/mL, or even lower than 150 RU/mL, the patient would require higher doses of rituximab, or repeated treatments, or alternative or combined therapy with one of the above immunosuppressants listed as examples but not limited to, or hemodialysis.

Typically, when the patient is considered as a good responder to rituximab (iCR and optionally non-iDom patients when immunodominance is assessed by competition assay), standardized doses of rituximab would be administered and found to be effective.

Typically, when the patient has an anti-PLA2R1 titer higher than 300 RU/mL, notably higher that 250 RU/mL, or higher that 200 RU/mL, rituximab treatment might be less effective and independent of the type of immunodominance, requiring either higher doses of rituximab or repeated treatments, or preferably alternative or combined immunosuppressive therapies as above. Typically, cyclophosphamide may be administrated.

### Method for the Treatment of Membranous Nephropathy

The present invention relates, in a fourth aspect, to a method for the treatment of membranous nephropathy in a subject in need thereof comprising:
the determination of PLA2R1 immunodominance in a sample obtained from a patient suffering from membranous nephropathy, comprising a step of determining the nature of the antibody which mostly drives the humoral response in said sample, according to any of the methods described above (i.e., competition assay or titer ratio) or others for determining immunodominance according to the present invention and
administering an effective amount of the immunosuppressant or an effective amount of a symptomatic treatment to said patient, when said patient is immunodominant for CysR or is non-immunodominant and thus considered as a good responder to a symptomatic treatment or immunosuppressant;
repeating the effective amount of the immunosuppressant or administering an effective amount of an alternative or combined stronger immunosuppressive therapy to said patient, or initiating an hemodialysis when said patient is immunodominant for CTLD1 and thus is resistant to immunosuppressant;

In some embodiment, the method comprises a step of selecting patients having an anti-PLA2R1 titer lower than 300 RU/mL, notably lower that 250 RU/mL, lower that 200 RU/mL, or even lower than 150 RU/mL.

By an “effective amount” of an immunosuppressant is meant a sufficient amount to treat membranous nephropathy, at a reasonable benefit/risk ratio applicable to any medical treatment. It is understood, however, that the total daily usage of the immunosuppressant is decided by the attending physician within the scope of sound medical judgment. The specific therapeutically effective dose for any particular subject in need thereof depend upon a variety of factors including the clinical and histopathological stage of membranous nephropathy, the activity of the immunosuppressant employed, the PLA2R1 immunodominance, the age, body weight, general health, sex and diet of the subject, the time of administration, route of administration, the duration of the treatment; drugs used in combination or coincidental with the and like factors well known in the medical art and clinical practice, including co-morbidities and associated diseases such as cancers and infections or other autoimmune diseases. For example, it is well known within the skill of the art to start doses of the compound at levels lower than those required to achieve the desired therapeutic effect and to gradually increase the dosage until the desired effect is achieved.

The term “treatment” or “method of treating” or its equivalent is not intended as an absolute term and, when applied to, for example, membranous nephropathy, refers to a procedure or course of action that is designed to reduce or eliminate or to alleviate one or more symptoms of membranous nephropathy.

Often, a “treatment” or a “method of treating” membranous nephropathy will be performed even with a low likelihood of success but is nevertheless deemed to induce an overall beneficial effect. Treatment of membranous nephropathy refers, for example, to delay of onset, reduced frequency of one or more symptoms, or reduced severity of one or more symptoms associated with the disorder. In some circumstances, the frequency and severity of one or more symptoms is reduced to non-pathological levels. More particularly, the term of “treatment” or a “method of treating” of membranous nephropathy refers to an improvement of clinical behavioral or biological criteria in the subject, including any clinical signs of partial or complete remission of membranous nephropathy (proteinuria, serum creatinine level, eGFR, etc).

Typically, the treatment or the method of treating could refer to a lower risk of requiring hemodialysis and/or a low risk of developing kidney failure. It also could refer to the fact that the subject would not require a stronger but more aggressive immunosuppressant. It could also refer to normalized or lowered levels of proteinuria and/or serum creatinine or a normalized or increased level of estimated glomerular filtration rate (eGFR).

Hence, the inventors demonstrate that immunodominance can be used as a biomarker per se and also as an additional clinical biomarker which can be combined with anti-PLA2R1 titer to help refine clinical outcome and likelihood of response to treatment.

In particular, stratifying patients according to immunodominance can be useful to guide and optimize therapy with different regimens of rituximab, for a better likelihood of response to treatment.

From a pathophysiological point of view, iC1 patients differ from iCR/non-iDom patients by exhibiting two main classes of immunodominant autoantibodies targeting PLA2R1 on both CysR and CTLD1 domains.

The immunodominance profile of iC1 patients may result from different genetic backgrounds and/or a more advanced autoimmune response, may be associated with more severe podocyte injury and larger immune deposits, and more resistance to immunosuppressive therapy.

The invention will be further illustrated by the following examples. However, these examples should not be interpreted in any way as limiting the scope of the present invention.

### EXAMPLES

In the following examples, the two major objectives were to i) provide a comprehensive analysis of the anti-PLA2R1 humoral response, focusing on the dissection of conformational PLA2R1 epitope-containing domains recognized by circulating autoantibodies from a large retrospective cohort of 142 patients with PLA2R1-associated MN (examples 1 and 2); and ii) evaluate how the specific characteristics of individual anti-PLA2R1 response observed among patients may be translated to the clinics to predict clinical outcome and response to therapy (example 3).

### Methods

Patients — A cohort of 142 patients was established with biopsy-proven primary MN by inclusion of patients from several French Departments of Nephrology. All included patients were not treated with immunosuppressive agents within 12 months prior to baseline serum sampling. Baseline serum samples were collected with a median time of 5 months (IQR 0-10 months) from kidney biopsy. eGFR was calculated according to the Chronic Kidney Disease Epidemiology Collaboration (CKD-EPI) formula. Median time to last follow-up was 26 months after sampling (IQR 23-58 months). The clinical outcome was analyzed according to the 2012 KDIGO recommendations after first-line therapy (NIAT (conservative therapy) or immunosuppressants (with rituximab given to most patients, 43% of the whole cohort)) with a median follow-up time of 12 months (IQR: 6-22 months) from baseline sampling. Partial remission was defined as proteinuria below 3.5 g/day and less than 50% of baseline value, accompanied by an increase or return to normal albuminemia and stable creatininemia. Complete remission was defined as proteinuria lower than 0.5 g/day and normal albuminemia and creatininemia. Remissions were considered as spontaneous if they occurred without administration of immunosuppressive drugs during follow-up. Clinically active disease was defined as proteinuria above 3.5 g/day and/or serum creatinine increase over 30% compared to baseline in the absence of any other cause. The study was approved by institutional review boards and conducted according to the principles of the Declaration of Helsinki. Written informed consent was obtained from all participants.

### Generation and Expression of PLA2R1 Mutants and Membrane-Bound Chimeras of PLA2R1-MRC2

All PLA2R1 domains and fragments as referred below are defined in reference to the complete human PLA2R1 protein sequence (reference Uniprot Q13018 shown as SEQ ID NO19). All soluble and membrane-bound PLA2R1 mutants as well as chimeras were generated by PCR and cloned into the pcDNA3.1/Zeo (-) expression vector (Life Technologies, Carlsbad, USA). Soluble and membrane-bound PLA2R1 constructs were generated using the Phusion Site-Directed Mutagenesis Kit (Thermo Fisher Scientific, Waltham, USA). Membrane-bound and soluble chimeras between MRC2 (Uniprot Q9UBG0) and PLA2R1 were generated using recombination-assisted megaprimer cloning essentially as described (Mathieu, J, Alvarez, E, Alvarez, PJ: Recombination-assisted megaprimer (RAM) cloning. MethodsX, 1: 23-29, 2014).

A series of constructs containing the CysR-FnII-CTLD1 triple domain of PLA2R1 (Q36 to N359 - constructs A to G except D) with and without various protease cleavage sites were designed based on previous work of Kao et al. (Kao L, Lam V, Waldman M, Glassock RJ, and Zhu Q. Identification of the immunodominant epitope region in phospholipase A2 receptor-mediating autoantibody binding in idiopathic membranous nephropathy. J Am Soc Nephrol. 2015;26(2):291-301). SEQ ID NO17 is shown as a representative example of these constructs. Constructs comprised the PLA2R1 signal peptide (M1 to A20) followed by its N-terminal linker sequence (E21 to W35), the N-terminal 6xHis and 3xFlag tags, the triple PLA2R1 domains with or without protease cleavage sites and a C-terminal HA-tag (except for construct F which was only HA-tagged). Protease cleavage sites were introduced at different amino acid positions as follows: construct A, no protease cleavage site; construct B, thrombin cleavage site (LVPRGS) inserted between CysR and FnII (replacing amino acids L166 to G171); construct C, thrombin cleavage site within the first disulfide bond of CTLD1 (replacing amino acids T231 to D236); construct D, same as construct C but with an additional factor Xa cleavage site (IEGR) within the linker region between FnII and CTLD1 (replacing amino acids T223 to E226); construct E, only factor Xa cleavage site (IEGR) within the linker region between FnII and CTLD1 (replacing amino acids T223 to E226); construct F, extended TEV protease cleavage site (GLENLYFQG) inserted in the linker region between FnII and CTLD1 (between D221 and P222), thereby extending the linker region between the two domains; construct G, thrombin cleavage site inserted in the linker region between FnII and CTLD1 (T223 to S224), thereby extending the region between the two domains. For construct D, a human codonoptimized synthetic gene was designed (Genecust, Dudelange, Luxembourg). The synthetic gene comprises the signal peptide of human group IIA secreted phospholipase A2 (M1 to N20, Uniprot P14555) which has been shown to drive high expression level of various proteins (Valentin, E, Ghomashchi, F, Gelb, MH, Lazdunski, M, Lambeau, G: On the diversity of secreted phospholipases A2. Cloning, tissue distribution, and functional expression of two novel mouse group II enzymes. J Biol Chem, 274: 31195-31202, 1999) followed by the N-terminal linker sequence of PLA2R1 (E21 to W35), N-terminal 6xHis and 3xFlag tags, the triple domain CysR-FnII-CTLD1 (Q36 to H377) with factor Xa and thrombin cleavage sites and a C-terminal HA tag.

All other soluble and membrane-bound PLA2R1 constructs refer to the complete human PLA2R1 protein sequence (reference Uniprot Q13018 shown as SEQ ID NO19) and comprised the PLA2R1 signal peptide (M1 to A20) followed by its N-terminal linker sequence (E21 to W35) and the human PLA2R1 sequence coding for the different PLA2R1 recombinant proteins: soluble PLA2R1 (Q36 to S1397, full extracellular domain), CTLD2-8 (C2-C8: Y-357 to S1397), CTLD2-6 (C2-C6: Y357 to P1114), CTLD3-5 (C3-C5: V507 to S979), CTLD6-8 (C6-C8: K947 to S1397), CTLD6-7 (C6-C7: K947 to L1246)1, CTLD7-8 (C7-C8: E1097 to S1397), CysR (CR: Q36 to K164), FnII (H163 to G228), CTLD1 (C1: P222 to A375), CTLD2 (C2: H360 to E512), CTLD3 (C3: V507 to F661), CTLD4 (C4: N649 to W809), CTLD5 (C5: K797 to T957), CTLD6 (C6: K947 to P1114), CTLD7 (C7: E1097 to L1246), CTLD8 (C8: P1235 to S1397), Δ7 (P1235 to Q1463, membrane-bound CTLD8). All recombinant proteins were C-terminally HA-tagged (YPYDVPDYA). Soluble PLA2R1, CR, FnII, C1, C2, C3, C4, C5, C8 and Δ7 constructs were also N-terminally 3x-Flag-tagged (DYKDDDDK); soluble PLA2R1, CR, FnII, C1, C2, C3, C4 and C5 were also N-terminally 6x-His-tagged. The SEQ ID NO10 to NO16 and NO18 are shown as a representative examples of the above constructs for soluble and membrane-bound fragments of PLA2R1 with tags.

PLA2R1/MRC2 chimeras were produced in the open reading frame of membrane-bound mature MRC2 protein (G31 to E1479). The CysR or CTLD1 domain of MRC2 was replaced by the corresponding domain of PLA2R1 (E21 to H167 for CysR and D221 to H377 for CTLD1). Soluble chimeras of the CTLD6-CTLD7 region from MRC2 (T956 to H1258) were constructed by replacing either CTLD6 or CTLD7 with the corresponding domain from PLA2R1 (W943 to D1111 and T1102 to P1244, respectively). All constructs were prepared in pcDNA3.1/Zeo (-) expression vector with a PLA2R1 signal peptide and were C-terminally HA-tagged and N-terminally 6xHis- and 3xFlag-tagged.

After sequencing of all cDNA constructs, the expression plasmids were transfected into HEK293 cells using a homemade calcium phosphate transfection kit ( Seitz-Polski, B, Dolla, G, Payre, C, Girard, CA, Polidori, J, Zorzi, K, Birgy-Barelli, E, Jullien, P, Courivaud, C, Krummel, T, Benzaken, S, Bernard, G, Burtey, S, Mariat, C, Esnault, VL, Lambeau, G: Epitope Spreading of Autoantibody Response to PLA2R Associates with Poor Prognosis in Membranous Nephropathy. J Am Soc Nephrol, 27: 1517-1533, 2016). or Exgen (Biomol GmbH, Hamburg, Germany). HEK293 cells were cultured in DMEM medium containing 1% penicillin/streptomycin solution and 10% heat-inactivated FBS (all from Gibco, Waltham, USA) at 37° C. in a humidified atmosphere of 5% CO₂. Transfected cells were selected with 0.2 mg/mL Zeocin (InvivoGen, San Diego, USA). For large-scale production of recombinant proteins, single clones or mixed populations stably selected were cultured to sub-confluency in complete medium at 37° C., then switched to serum-free medium (OptiMEM) and incubated at 37° C. For PLA2R1 constructs with low expression at 37° C., cells were grown at 30° C. with or without tauroursodeoxycholic acid (TUDCA, Sigma-Aldrich, St. Louis, USA) or 4-phenyl butyric acid (PBA, Sigma-Aldrich, St. Louis, USA) to enhance expression, trafficking and/or folding of recombinant proteins as previously described for various mutated proteins. After seven days of expression, cell culture medium was collected and cells were washed with PBS, scrapped and lyzed in 20 mM Tris pH 7.4, 2 mM EDTA and protease inhibitor cocktail (Roche Diagnosis, Basel, Swizterland). Cells were sonicated and centrifuged in a microcentrifuge apparatus at 14,000 rpm for five minutes at 4° C. The supernatant corresponding to the cytosolic fraction (CF) was collected and the pellet resuspended and solubilized in 50 mM Tris pH 7.4, 2 mM EDTA, 100 mM NaCl, 1% Triton X-100, 0.5% sodium deoxycholate with protease inhibitors. The suspension was sonicated, incubated for one hour at 4° C. under rotation and centrifuged for 15 minutes at 14,000 rpm at 4° C. The supernatant corresponding to the solubilized fraction (SF) was recovered. Total protein concentration of each fraction was determined by BCA protein assay (Thermo Fisher Scientific, Waltham, USA). When the recombinant proteins were expressed at very low levels, culture medium was either precipitated with trichloroacetic acid (TCA) using standard protocol or purified by affinity chromatography on complete His-tag beads according to the manufacturer’s protocol (Roche, Basel, Switzerland). Eluted purified proteins were concentrated and buffer-exchanged with Fxa buffer (50 mM Tris pH 8.0, 1 mM CaCl₂, 10 mM NaCl) and 5 mM N-dodecyl-N-N-dimethyl-3-ammonio-1-propanesulfonate (SB12) using a centricon centrifugal filter device (Amicon, Millipore, Bedford, USA) equipped with an YM-30 membrane.

### Protease Digestion of the Triple Domains CysR-FnII-CTLD1 Constructs

Purified protein or culture medium from constructs B to G were digested overnight at 37° C. with thrombin (Thr, Calbiochem, San Diego, USA), factor Xa (F_{Xa}, Amersham Biosciences, UK) or tobacco etch virus (TEV, Sigma-Aldrich, St. Louis, USA) proteases according to purchasers’ recommendations. Cleaved products were immunodetected by WB.

### Immunodetection of PLA2R1 Recombinant Proteins

Recombinant proteins were run on SDS-PAGE gels under reducing or non-reducing conditions as originally described by Laemmli (Laemmli, UK: Cleavage of structural proteins during the assembly of the head of bacteriophage T4. Nature, 227: 680-685, 1970). Proteins were transferred to a methanol-soaked poly-vinylidene difluoride membrane (PerkinElmer, Waltham, USA) under semi-dry conditions (25 mM Tris, 192 mM glycine, pH 8.5, 20% ethanol) using Trans-blot Turbo (Bio-rad laboratories, Hercules, USA) at 25 V constant for 30 minutes. Culture medium containing the proteins of interest was slot-blotted onto a nitrocellulose membrane (Whatman, Maidstino, UK). For both western and slot blots, non-specific binding was blocked with 5% (w/v) low-fat dry milk in PBS-Tween 0.05% for one hour or overnight at 4° C. Membranes were then incubated with primary antibody under agitation for two hours or overnight at 4° C. After three washes for five minutes each, the membranes were incubated with horseradish peroxidase (HRP)-conjugated secondary antibody, under agitation for one hour. The membranes were washed three times for five minutes and the immunoreactive bands were detected with enhanced chemiluminescence substrate (ECL, PerkinElmer, Waltham, USA) and a Fusion-FX digital imager.

### Immunoprecipitation of PLA2R1 Epitope-Containing Domains

Proteins of interest were pulled-down from cell culture medium with MN patients’ serum overnight at 4° C. followed by incubation with anti-IgG4 affinity beads (Thermo Fisher Scientific, Waltham, USA) for one hour at 4° C. After three washes with Tris-buffered saline (TBS+: 20 mM Tris/HCl pH 7.2, 150 mM NaCl, 5 mM CaCl₂) and centrifugation in a microcentrifuge apparatus at 14,000 rpm for 15 minutes at 4° C., bound protein was eluted with 2× Laemmli buffer and analyzed by WB.

Immunoblotting of PLA2R1 recombinant proteins — Recombinant proteins were analyzed by WB under reducing or non-reducing conditions or by dot-blot. Primary antibodies (mouse monoclonal anti-HA antibody (1:5,000, Sigma-Aldrich, St. Louis, USA), mouse monoclonal anti-Flag antibody (1:1,000, Sigma-Aldrich, St. Louis, USA) and MN patients’ serum (1:100 unless stated otherwise) were all diluted in 0.5% low-fat milk in PBS-Tween 0.05% while secondary antibodies (anti-mouse antibody (1:20,000, Cambridge, UK) and anti-human IgG4 (1:7,500, Southern Biotech, Birmingham, USA)) were diluted in PBS-Tween 0.05%.

ELISA assays — For HA-based antigen capture ELISA assays, 96-well microplates (Thermo Fisher Scientific, Waltham, USA) were coated with anti-HA antibody (1:5,000, Sigma-Aldrich, St. Louis, USA) diluted in 20 mM Tris pH 8.0 overnight at 4° C. Plates were blocked with SeramunBlock (Seramun Diagnostica GmbH, Heidesee, Germany) for two hours and then washed with PBS-Tween 0.05%. HA-tagged PLA2R1 antigens (10-100 µl of cell culture medium diluted in PBS) were captured by incubation for two hours and then washed. Patients’ sera diluted in 0.1% (m/v) low-fat dry milk in PBS were added to wells and incubated for two hours. Plates were then washed and incubated for one hour with anti-human IgG4 horseradish peroxidase (HRP)-conjugated secondary antibody (1:7,500, Southern Biotech, Birmingham, USA) diluted in SeramunStab ST (Seramun Diagnostica GmbH, Heidesee, Germany). After three washes, tetramethylbenzidine peroxidase substrate (TMB, Interchim, Montluçon, France) was added and developed for 15 minutes before stopping the reaction with 1.2 N HCl. Optical density (OD) was read at 450 nm using a Multiskan FC plate reader (Thermo Scientific, Waltham, USA). Serum-free medium from mock-transfected HEK293 cells was used as a negative control for each patient, providing individual background. Cut-off OD values were determined as twice the background value for each individual patient.

Autoantibody titers for full PLA2R1, CysR and CTLD1 domains were determined by performing ELISA in which a standard curve was added. The standard curve consisted of seven dilutions of a highly PLA2R1-, CysR- or CTLD1-positive serum, allowing the conversion of optical density into RU/mL using a 5-parameter logistic curve (GraphPad Prism 7 Software, San Diego, USA). Anti-PLA2R1 titer was also measured with anti-total IgG using the commercial standardized ELISA from Euroimmun (Medizinische Labordiagnostika AG, Lübeck, Germany, Dahnrich, C, Komorowski, L, Probst, C, Seitz-Polski, B, Esnault, V, Wetzels, JF, Hofstra, JM, Hoxha, E, Stahl, RA, Lambeau, G, Stocker, W, Schlumberger, W: Development of a standardized ELISA for the determination of autoantibodies against human M-type phospholipase A2 receptor in primary membranous nephropathy. Clin Chim Acta, 421C: 213-218, 2013).

For ELISA competition assays, microplates (Thermo Fisher Scientific, Waltham, USA) were coated overnight at 4° C. with the purified full extracellular domain of PLA2R1 (10 ng diluted in 20 mM Tris pH 8.0) expressed as a recombinant secreted protein from HEK293 cells as described (Dahnrich, C, Komorowski, L, Probst, C, Seitz-Polski, B, Esnault, V, Wetzels, JF, Hofstra, JM, Hoxha, E, Stahl, RA, Lambeau, G, Stocker, W, Schlumberger, W: Development of a standardized ELISA for the determination of autoantibodies against human M-type phospholipase A2 receptor in primary membranous nephropathy. Clin Chim Acta, 421C: 213-218, 2013), then blocked with SeramunBlock (Seramun Diagnostica GmbH, Heidesee, Germany) for two hours and washed with PBS-Tween 0.05%. Patients’ sera were preincubated for 30 minutes with cell culture medium containing saturating amounts of different recombinant proteins (PLA2R1, CysR-FnII-CTLD1, CysR, CTLD1, CTLD5, CTLD7, CysR+CTLD2-8, CTLD1+CTLD2-8, or mock as a negative control), then added to wells coated with full PLA2R1 antigen and incubated for two hours. Preliminary dose-response experiments were performed to determine the appropriate patients’ serum dilutions as well as the volumes of cell culture medium sufficient to ensure full competition for each recombinant protein (data not shown). The plates were then washed, incubated with secondary antibody and revealed as described above. Results of competition assays are expressed as the percentage of maximal signal measured in the absence of competitor, taking into account the non-specific signal for individual patient.

Patients were classified as immunodominant for CysR (iCR) when competition with CysR-FnII-CTLD1 was higher than 65% while competition with CTLD1 was lower than 20%. Patients were classified as immunodominant for CTLD1 (iC1) when competition with Cys-FnII-CTLD1 was higher than 65% while competition with CTLD1 was higher than 20%. Patients were classified as non-immunodominant (non-iDom) when competition with CysR-FnII-CTLD1 was lower than 65% while competition with any individual domain was too low to determine any specific immunodominance, meaning that no specific domain was driving the signal of the humoral autoimmune response. For 17 of 142 patients, serum was not available to perform competition assays. Among them, 11 were positive only for the CysR domain, and assigned as iCR. The last 6 patients were positive for CysR and other epitope-containing domains, and no immunodominant profile could be assigned. These 6 patients were excluded from analyses of immunodominance.

Statistical analysis — Patients characteristics are presented as frequencies and percentages for qualitative variables, and medians and interquartile ranges for continuous variables. Wilcoxon-Mann-Whitney or Kruskal-Wallis rank tests were used to assess the relationship between continuous and qualitative variables; Pearson Chi-Squared or Fisher Exact tests for qualitative variables and Spearman rank correlation for continuous variables. Unadjusted and adjusted analyses were performed using logistic regression. Selection of variables for multivariate analysis was based on a threshold at 0.20. As age, gender and serum creatinine were linked, we choose to use eGFR in multivariate analyses. Treatment and multivariate analyses were also adjusted for proteinuria, anti-PLA2R1 titer according to median level and treatment. Two variables were built combining treatment and anti-PLA2R1 titer according to median level or immunodominance, respectively, and were used in a second model to assess the interaction with treatment. Data are expressed among the population with no missing value for eGFR, proteinuria, treatment, anti-PLA2R1 titer and immunodominance. Hosmer and Lemeshow test was used to assess goodness of fit. All tests were two-sided and p-values <0.05 indicated statistical significance. Analyses were performed using SAS v.9.3 software (SAS Institute, Cary, USA) or GraphPad Prism 7 Software (San Diego, USA).

### Example 1: Anti-PLA2R1 Autoantibodies Can Target Up to 5 Independent Epitope-Containing Domains Spreading All Over the Extracellular Region of PLA2R1

### CysR and CTLD1 Are Two N-terminal Independent Epitope-Containing Domains

The inventors first clarified the controversy between overlapping versus independent epitopes within the N-terminal CysR-FnII-CTLD1 region by building up on the approach based on site-directed mutagenesis coupled to serial insertion of thrombin cleavage sites between domains, as originally described by Kao and coworkers (Kao, L, Lam, V, Waldman, M, Glassock, RJ, Zhu, Q: Identification of the immunodominant epitope region in phospholipase A2 receptor-mediating autoantibody binding in idiopathic membranous nephropathy. J Am Soc Nephrol, 26: 291-301, 2015). Importantly, the inventors noticed that in the original 1-2T construct from Kao et al., the thrombin cleavage site was inserted within the first disulfide bond of CTLD1 and not in the small linker region between FnII and CTLD1 (Kao L, Lam V, Waldman M, Glassock RJ, and Zhu Q. Identification of the immunodominant epitope region in phospholipase A2 receptor-mediating autoantibody binding in idiopathic membranous nephropathy. J Am Soc Nephrol. 2015;26(2):291-301).

In total, seven soluble constructs expressing CysR-FnII-CTLD1 recombinant proteins with N-terminal 6xHis/3xFlag and C-terminal HA tags, and harboring either no protease cleavage site, or different protease cleavage sites between PLA2R1 domains have been produced. The recombinant proteins were cleaved by proteases and the WB reactivity of sera from MN patients with different epitope profiles determined by indirect ELISA were tested. Construct B harbors a thrombin cleavage site between CysR and FnII and provided straightforward results. It clearly shows that patients’ sera react with independent epitopes on the thrombin-cleaved fragments CysR and FnII-CTLD1 domains, a finding in contrast with previous data from Kao et al. suggesting an overlapping epitope. The inventors next produced construct C as described by Kao et al. In inventors’ hands, construct C was hardly expressed in HEK293 cells, likely because of insertion of the thrombin cleavage between the CTLD1 disulfide bond, which may produce a structural clash. Expression was rescued by growing cells at 30° C. and thrombin cleavage was effective, as shown by western blot detection of the cleaved fragments with anti-tag antibodies performed under reducing conditions. However, western blot analysis under non-reducing conditions showed no apparent cleavage, in fact because the two PLA2R1 fragments are maintained in a covalent manner by the CTLD1 disulfide bond, despite actual cleavage of the amino acid peptide bond by thrombin. As a result, serum from different MN patients could only react with the triple domain instead of any cleaved fragments under non-reducing conditions, a finding which could have been interpreted as an overlapping epitope between the different domains. To demonstrate that patients could recognize cleaved fragments from constructs similar to C but when the protease cleavage site is correctly inserted between FnII and CTLD1 domains, the inventors prepared several other constructs and end up with construct G where a thrombin cleavage site was inserted within an extended linker region between FnII and CTLD1. Construct G could be only partially cleaved by thrombin, but this was sufficient to demonstrate that patients’ sera had reactivity to either CysR-FnII or CTLD1 or both cleaved domains.

Collectively, the above results suggest the presence of distinct and independent epitopes in each of the CysR and CTLD1 domains without evidence of overlapping epitopes. To ascertain this finding and demonstrate that CysR and CTLD1 contain independent epitopes in the context of a full-size molecule, the inventors prepared two MRC2-PLA2R1 membrane-bound chimeras where the CysR and CTLD1 domains of PLA2R1 are individually introduced into the MRC2 backbone in place of the corresponding domains. MRC2 is the closest paralog of PLA2R1 and has the same overall structural organization (Engelholm LH, Ingvarsen S, Jurgensen HJ, Hillig T, Madsen DH, Nielsen BS, and Behrendt N. The collagen receptor uPARAP/Endo180. Front Biosci. 2009;14(2103-14.), but is not reactive with MN patients positive or not for PLA2R1 (Tomas NM, Beck LH, Jr., Meyer-Schwesinger C, Seitz-Polski B, Ma H, Zahner G, Dolla G, Hoxha E, Helmchen U, Dabert-Gay AS, et al. Thrombospondin type-1 domain-containing 7A in idiopathic membranous nephropathy. N Engl J Med. 2014;371(24):2277-87). The inventors validated the expression of chimeras versus wild-type paralogs and tested the reactivity of eight different MN patients. Both ELISA and WB data confirmed that patients could independently recognize CysR and/or CTLD1 domains when inserted into the MRC2 full-size molecule, in good accordance with their epitope profile. Finally, the FnII domain was tested as a possible independent epitope-containing domain after expression as a single domain and was found to be not reactive to all MN sera tested.

### CTLD5, CTLD7 and CTLD8 Are Three C-Terminal Independent Epitope-Containing Domains

To investigate whether the remaining large C-terminal region of PLA2R1 (CTLD2 to CTLD8) contains epitope-containing domains beyond the known CTLD7 domain, the inventors produced various PLA2R1 recombinant proteins spanning the CTLD2-CTLD8 region, validated their expression by WB and tested the reactivity of a subset of 28 MN patients by ELISA. Representative data have been collected for four patients having different reactivities to CTLD7 but also other CTLDs.

First, reactivity to CTLD7 as a single domain has been confirmed. Ten of the 28 patients (35.7%) were positive for CTLD7. In previous studies, CTLD6-7 appeared to be more reactive than CTLD7, especially by western blot analysis. This would suggest assisted folding of the CTLD7 conformational epitope by CTLD6 or the presence of additional epitopes in CTLD6. The inventors thus verified that CTLD7 but not CTLD6 was reactive as an independent epitope-containing domain when expressed as soluble chimeras between PLA2R1 and MRC2. The C6M-C7P chimera containing the PLA2R1 CTLD7 domain was clearly reactive while the C6P-C7M chimera containing the PLA2R1 CTLD6 domain was not reactive, indicating reactivity restricted to CTLD7.

Interestingly, among the 28 patients, additional reactivities to CTLD2-6 for seven patients (25%) and to CTLD8 for two patients (7.1%) were found, suggesting other epitopes. To further investigate the reactivity within the CTLD2-6 region and confirm CTLD8 as an independent epitope-containing domain, a series of single domains from CTLD2 to CTLD8 as well as Δ7, the membrane-bound form of CTLD8, were produced. Among the four above representative patients, the three reacting to CTLD2-6 also recognize CTLD5 but none of the other single domains within this region by ELISA. To confirm these results by another technique, the reactivity of patients against the same recombinant proteins by WB was tested. CTLD2-8, but not CTLD2-6 and CTLD3-5 was reactive, despite efficient transfer of all three proteins under non-reducing conditions. When expressed as single domains, CTLD3, CTLD5 and Δ7 but not CTLD2, CTLD4, CTLD6, CTLD7 and CTLD8 were efficiently transferred under non-reducing conditions, but none of these domains was recognized by patients. WB performed with improved transfer conditions and lower serum dilutions showed reactivity to CTLD8 but not CTLD5. Together, the above data suggest that patients’ sera 1) are reactive to CTLD2-8 by WB because of reactivity to CTLD7 and/or CTLD8 but not CTLD5 (with CTLD2-8 allowing efficient transfer of CTLD7 and CTLD8) and 2) are not reactive to CTLD5 by WB when presented either as a single domain or as CTLD2-6 and CTLD3-5 larger fragments (with all three proteins transferring efficiently). This indicates that CTLD5 is reactive only in its native conformation, i.e. by ELISA but not WB. Interestingly, we observed that the CTLD5 reactivity by ELISA was more sensitive to temperature and DTT than the other CTLDs or the CysR domain. Further analysis by immunoprecipitation and dot-blot confirmed the reactivity of patients to CTLD5 and CTLD8, identifying them as two novel independent epitope-containing domains. Conversely, neither CTLD2, CTLD3, CTLD4 nor CTLD6 were recognized by patients by ELISA or immunoprecipitation.

### Example 2: Prevalence, Immunodominance and Relationship With Anti-PLA2R1 Titer

Collectively, the above findings indicate that the autoimmune response against PLA2R1 is polyclonal and leads to the presence of multiple circulating anti-PLA2R1 autoantibodies targeting up to five distinct PLA2R1 domains that spread the entire extracellular region from the N-terminal to the C-terminal ends. Interestingly, all epitopes were found to be conformational, including the novel epitopes present in CTLD5 and CTLD8. The next question was the prevalence and immunodominant properties of the five epitope-containing domains relative to the anti-PLA2R1 titer as measured with the standardized ELISA.

### CysR and CTLD5 Are the Most Prevalent Epitope-Containing Domains

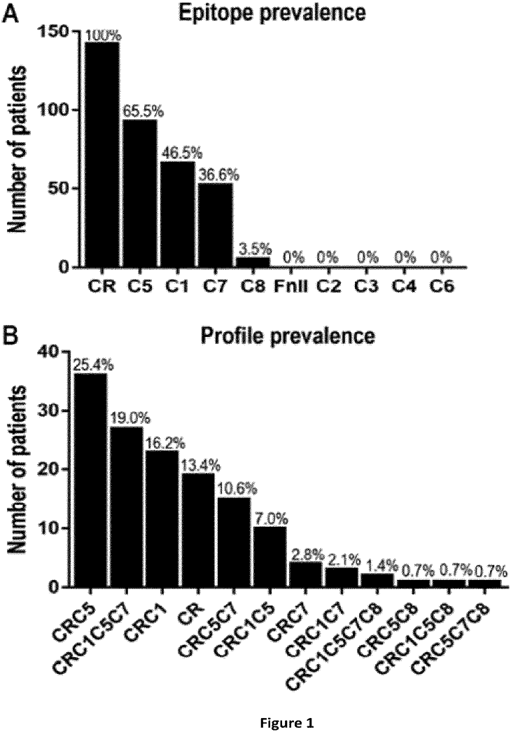

To determine which epitope-containing domains are most prevalent, the inventors screened a retrospective cohort of 142 PLA2R1-positive MN patients against the 10 single PLA2R1 domains by ELISA. As expected, the five epitope-containing domains were recognized with different prevalence (FIG. 1A). None of the other single domains, namely FnII, CTLD2, CTLD3, CTLD4 and CTLD6 were recognized. CysR was recognized by all patients (FIG. 1A), thereby appearing as the most prevalent epitope-containing domain. Interestingly, CTLD5 was the second most prevalent domain with 65.5% reactivity followed by CTLD1 (46.5%), CTLD7 (36.6%) and CTLD8 (3.5%) (FIG. 1A). Based on the combined prevalence, patients could be stratified into different epitope profiles, with CRC5 being the most abundant (FIG. 1B).

### Epitope Positivity Increases With Anti-PLA2R1 Titer

To analyze the relationship between epitope positivity and anti-PLA2R1 titer in the cohort, patients were ranked by titer as measured with the standardized ELISA (Dahnrich C, Komorowski L, Probst C, Seitz-Polski B, Esnault V, Wetzels JF, Hofstra JM, Hoxha E, Stahl RA, Lambeau G, et al. Development of a standardized ELISA for the determination of autoantibodies against human M-type phospholipase A2 receptor in primary membranous nephropathy. Clin Chim Acta. 2013;421C(213-8.)) and the positivity for each epitope-containing domain was plotted (FIG. 2A). While all patients were positive for CysR, CTLD5-positive patients were distributed over the full range of anti-PLA2R1 titer. In contrast, CTLD1-positive patients were more present at high titers, even though some are present in the first tertile. CTLD7-positive patients were also more abundant in the second and third tertiles of anti-PLA2R1 titer, and the rare CTLD8-positive patients were only found at medium to high titers. Overall, anti-PLA2R1 titer increases as the number of positive epitope-containing domains also increases. Finally, when patients were ranked by epitope prevalence and the complexity of their epitope profile while considering positivity or not for CTLD 1, a relationship between the complexity of epitope profile and the increase in anti-PLA2R1 titer was observed (FIG. 2B).

### CvsR and CTLD1 Are the Immunodominant Epitope-Containing Domains

Beyond prevalence, the inventors aimed to determine which PLA2R1 domains contain the major immunodominant epitopes that would contribute to most of the signal measured by ELISA on the full PLA2R1 antigen. Towards this aim, competition ELISA assays between full PLA2R1 (complete extracellular region) as target antigen and various PLA2R1 recombinant proteins as competitors (PLA2R1, CysR-FnII-CTLD1, CysR, CTLD1, CTLD5, CTLD7, mix of CysR and CTLD2-8, and mix of CTLD1 and CTLD2-8) were developed. In these experiments, patients’ sera were first preincubated with an excess of competitor and then tested against full PLA2R1 antigen to measure the remaining anti-PLA2R1 reactivity. As expected, competition with homologous full PLA2R1 was total for all patients’ sera, validating assay conditions. For the majority of patients, the N-terminal CysR-FnII-CTLD1 triple domain could inhibit from 65 to 100% of the PLA2R1 signal, indicating that the autoimmune response is mostly driven by autoantibodies targeting CysR and/or CTLD1 epitope-containing domains. Further competition with CysR or CTLD1 as single domains as well as with CysR or CTLD1 mixed with CTLD2-8 demonstrated that both CysR and CTLD1 are major contributors of anti-PLA2R1 reactivity. In contrast, the C-terminal CTLD5 and CTLD7 epitope-containing domains appeared as minor contributors for most patients. Competition assays for CTLD8 were not performed because of its low prevalence and likely minor contribution, as inferred from competition data measured with single CTLD5 and CTLD7 domains versus CysR and CTLD1 domains mixed or not with the CTLD2-8 region.

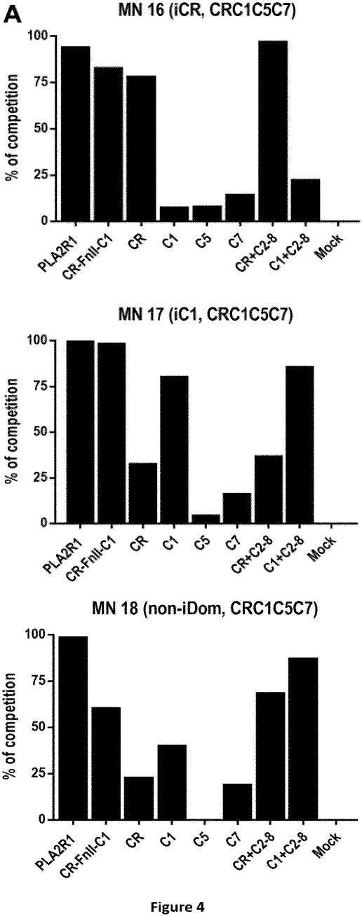

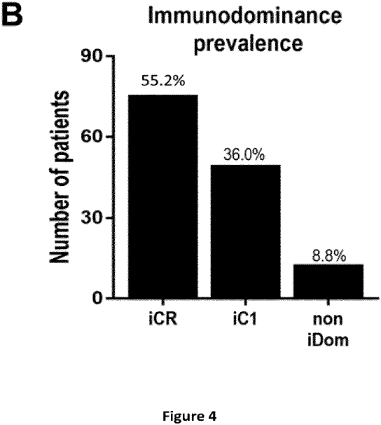

The above experiments indicate that patients’ sera exhibit different patterns of circulating autoantibodies based on epitope positivity and competition profiles, with CysR and CTLD1 clearly containing the immunodominant epitopes and driving the humoral autoimmune response for most patients. Taking into account both epitope prevalence and competition assays, the inventors stratified patients into three groups: immunodominant CysR (iCR), immunodominant CTLD1 (iC1) and non-immunodominant (non-iDom). iCR patients are defined by a humoral response mostly driven by autoantibodies targeting CysR (contributing to 65-100% of the PLA2R1 signal reactivity) and a low contribution of autoantibodies recognizing other epitope-containing domains including CTLD1. iC1 patients are defined by a humoral response driven not only by anti-CysR but also by anti-CTLD1 autoantibodies, with these latter contributing up to 80% of the PLA2R1 signal reactivity (i.e. with a balanced and gradual increase of anti-CTLD1 reactivity at the expense of anti-CysR reactivity), and little contribution from other distal epitope-containing domains. Non-iDom patients are defined by a humoral response where the PLA2R1 signal appears to be uniformly spread over the different epitope-containing domains without indication of immunodominance by a particular epitope domain. The patient’s stratification is further defined in methods and illustrative patients’ cases from each group are shown in FIG. 4A. Interestingly, patients having the same epitope profile (for instance CRC1C5C7) can belong to different immunodominant groups (FIG. 4A). As shown in FIG. 4B, the majority of patients was iCR (55.2%) while a significant number was iC1 (36.0%), and a minority was non-iDom (8.8%).

### Anti-PLA2R1 Titer and Immunodominance

As above, patients were ranked by anti-PLA2R1 titer as measured by the standardized ELISA and their immunodominant profile was displayed (FIG. 3A). The inventors observed that iCR patients were more present in the first and second tertiles of anti-PLA2R1 titer while iC1 patients were inversely more present in the third tertile, and non-iDom patients were present in the first and second tertiles but absent in the third tertile (FIG. 3A). When combining epitope positivity and immunodominance, non-iDom, iCR and iC1 patients can be ranked according to the increasing complexity of their epitope profiles, and it can be observed that the anti-PLA2R1 titer increases as epitope positivity develops towards the C-terminal region of PLA2R1 up to CTLD7 and CTLD8, for both types of immunodominance (FIG. 3B). Interestingly, the few non-iDom patients had relatively low anti-PLA2R1 titer despite having complex epitope profiles up to CTLD7, and could be best positioned between the iCR and iC1 groups (FIG. 3B). This supports the view that the highest anti-PLA2R1 titers (third tertile) can only be observed when CysR and/or CTLD1 act as immunodominant epitope-containing domains and drive most of the anti-PLA2R1 signal.

### Correlation Between Anti-PLA2R1 Titers for Full PLA2R1 and Immunodominant Epitope-Containing Domains

Since CysR and CTLD1 were identified as the immunodominant epitope-containing domains, the inventors measured their titer versus full PLA2R1 antigen for all patients (detection of anti-PLA2R1 IgG4), and analyzed the correlations with anti-PLA2R1 titer measured with the standardized ELISA (detection of anti-PLA2R1 total IgG). Highly positive correlations were observed between anti-PLA2R1 titers measured for full PLA2R1 with total IgG versus IgG4 when analyzing the cohort as a whole (r=0.89) or after stratification into iCR or iC1 patients (r=0.85 and r=0.86, respectively) (FIG. 5, top panels). Highly positive correlations were also observed between full anti-PLA2R1 titer (total IgG) and anti-CysR titers for the whole population as well as iCR and iC1 patients (r=0.82, r=0.81 and r= 0.90, respectively, FIG. 5, middle panels). However, the correlation between full anti-PLA2R1 titer and anti-CTLD 1 titer was higher for iC 1 patients (r=0.80) than for the full cohort or iCR patients (r=0.58 and r=0.50, respectively, FIG. 5, lower panels). Similar observations were made for correlations between full anti-PLA2R1 titer with detection of IgG4 antibodies and anti-CysR or anti-CTLD1 titers. Interestingly, non-iDom patients had anti-PLA2R1 titers for full PLA2R1, CysR and CTLD1 scattered in a relatively narrow range, with low to medium range values. As observed for anti-PLA2R1 titer, the anti-CysR and anti-CTLD1 titers increased as the number of positive epitope-containing domains increased. Finally, the inventors also analyzed the anti-PLA2R1, anti-CysR and anti-CTLD1 titers for the different immunodominant groups. Overall, median titers for the iCR and non-iDom groups were similar and lower than those for iC1 patients, suggesting an additional immunodominant effect of CTLD1 over CysR immunodominance.

In summary, from examples 1 and 2, the inventors specifically showed that in PLA2R1-positive MN patients: i) Circulating anti-PLA2R1 autoantibodies can recognize up to 5 epitope-containing domains, including CysR, CTLD1, CTLD5, CTLD7 and CTLD8, indicating that 50% of the PLA2R1 extracellular region is targeted by autoantibodies; ii) The epitope prevalence decreases from the N-terminal to the C-terminal epitope-containing domains; iii) The N-terminal CysR and CTLD1 domains harbor the major immunodominant epitopes which contribute to most of the anti-PLA2R1 titer measured by the standardized ELISA; and iv) The C-terminal domains CTLD5, CTLD7 and CTLD8 harbor non-immunodominant epitopes which collectively have a minor contribution to the anti-PLA2R1 titer as measured by the standardized commercial ELISA. Together, the overall humoral autoimmune response appears to be mostly driven by the N-terminal CysR and/or CTLD1 domains functioning as two key yet alternative immunodominant epitope-containing domains while the distal spreading to the C-terminal other domains contributes to little of the full anti-PLA2R1 titer.

The inventors further show that analysis of the ratio of anti-CTLD1/anti-CysR titers (for instance as determined by in-house IgG4 detection ELISA) can be used as an advantageous surrogate method of competition ELISA to determine immunodominance. The immunodominant profile of patients as determined by competition ELISA clearly correlates with the ratio (FIG. 7). In the study cohort, patients with a value of ratio below the median ratio of anti-CTLD1/anti-CysR titers were mostly iCR while patients above the median were mostly iC1. As expected, the authors observed that patients with a titer ratio below the median (thus iCR) have a higher anti-CysR titer and a lower anti-CTLD1 titer compared to patients above the median ratio (thus iC1), while no differences were observed in the total anti-PLA2R1 titer as measured with the standardized ELISA (FIG. 8).

### Example 3: Clinical Association With Immunodominance (Assessed By Competition Assay Or By Titer Ratio), Epitopes And Anti-Pla2r1 Titer

Having in hands the various features of anti-PLA2R1 circulating autoantibodies for 142 MN patients, the inventors analyzed their association with clinical presentation and outcome.

### Clinical Characteristics And Features Of Anti-Pla2r1 Autoantibodies

With a median age of 55 years, about two thirds of male patients and a high proteinuria, the baseline clinical characteristics of the study cohort were similar to other MN cohorts (Seitz-Polski, B, Dolla, G, Payre, C, Girard, CA, Polidori, J, Zorzi, K, Birgy-Barelli, E, Jullien, P, Courivaud, C, Krummel, T, Benzaken, S, Bernard, G, Burtey, S, Mariat, C, Esnault, VL, Lambeau, G: Epitope Spreading of Autoantibody Response to PLA2R Associates with Poor Prognosis in Membranous Nephropathy. J Am Soc Nephrol, 27: 1517-1533, 2016; Seitz-Polski, B, Debiec, H, Rousseau, A, Dahan, K, Zaghrini, C, Payre, C, Esnault, VLM, Lambeau, G, Ronco, P: Phospholipase A2 Receptor 1 Epitope Spreading at Baseline Predicts Reduced Likelihood of Remission of Membranous Nephropathy. J Am Soc Nephrol, 29: 401-408, 2018. Forty-three percent of patients received conservative treatment (NIAT) while 57% received immunosuppressants (IS, among which most had rituximab (75% of IS-treated patients representing 43% of the whole cohort). As for features of anti-PLA2R1 autoantibodies, anti-PLA2R1 titers against the PLA2R1 antigen were measured with the standardized ELISA for total IgG and homemade ELISA for IgG4. IgG4 titers for specific autoantibodies targeting CysR or CTLD1 domains were also measured, as they harbor the immunodominant epitopes and allowed the stratification of patients by immunodominance.

### Stratification by Immunodominance

For immunodominance, 55% of patients were classified as iCR, 36% iC1 and 9% non-iDom (FIG. 4B).

### Stratification by Epitope Profiling

For epitope profiling, besides the stratification of patients by prevalence for the five PLA2R1 epitope-containing domains and by positivity for the number of epitope-containing domains (irrespective of immunodominance), the inventors stratified patients based on the hypothesis of epitope spreading occurring during the maturation of the humoral autoimmune response from CysR to other domains such as CTLD1 and CTLD7, as described in the patent application WO/2017/009245.

In this hypothesis, at the time neither CTLD5 nor CTLD8 domains were known as epitope-containing domains, patients were stratified into those only positive for the CysR domain (also called non-spreaders, and now corresponding to the CR±C5±C8 patients (n=56)) and those positive for CysR with additional positivity for CTLD1 and/or CTLD7 (called spreaders, n=86). In the current hypothesis taking into account the identification of CTLD5 and CTLD8 as epitope-containing domains, the group of patients with a CysR only profile became lower (n=19) while the group of patients with spreading to “any other epitope” became larger (n=123). The association between the above antibody features to clinical presentation and clinical outcome was analyzed below.

### Clinical Presentation and Outcome According to Immunodominance

The clinical characteristics at baseline and clinical outcome after first-line therapy according to stratification of patients into the iCR, iC1 and non-iDom groups were first compared. Patients did not present any differences in age, gender or clinical parameters, except for proteinuria which was lower in iCR than iC1 patients (5.3 vs 6.8 g/day, p=0.02). iCR, non-iDom and iC1 patients did not differ for treatment with immunosuppressants (IS) versus conservative therapy (NIAT).

However, clinical outcome was significantly different among subgroups (FIG. 6A). The majority of iCR and non-iDom patients reached remission (64.0 and 75.0%, respectively) whereas the opposite was observed for iC1 patients, with the majority remaining in active disease or progressing to ESKD (59.2%). iC1 patients had higher anti-PLA2R1 titer at baseline (178.0 RU/mL) compared to iCR and non-iDom patients (56.5 RU/mL and 59.7 RU/mL, respectively). Positivity towards the various epitope-containing domains and spreading to CTLD1 and/or CTLD7 domains was more frequent in iC1 than iCR patients.

### Clinical Presentation and Outcome According to Epitope Profiling

The clinical characteristics at baseline and clinical outcome after first-line therapy were compared (FIGS. 6B and 6C) when considering the epitope profiling corresponding to our original (CR±C5±C8 versus positivity for CTLD1 and/or CTLD7, as described in the patent application WO/2017/009245) and current (CR only versus CR + any other epitope, taking into account the identification of CTLD5 and CTLD8 as novel epitope-containing domains) working hypothesis of epitope spreading.

Patients presenting autoantibodies only towards the CysR domain had better kidney function (89 vs 68 mL/min/1.73 m², p=0.0078), lower proteinuria (4.6 vs 6.4 g/day, p=0.0452) and lower autoantibody titers than patients having autoantibodies against CysR plus any other domain. However, the clinical outcome did not differ between patients with CysR only reactivity versus those with CysR plus any other epitope reactivity.

This observation contrasted with our original hypothesis where patients with CysR only reactivity had better clinical outcome than those with CysR plus additional reactivity to CTLD1 and/or CTLD7 (as described in WO/2017/009245).

### Exploration of Associations Between Baseline Characteristics and Clinical Outcome

The baseline clinical parameters and anti-PLA2R1 features that may differ between patients entering or not into remission were analyzed. Patients reaching remission were younger, had a better renal function and lower proteinuria at presentation. In agreement with previous studies, patients reaching spontaneous or IS-induced remission had a lower anti-PLA2R1 titer at baseline (FIG. 6D), differed in epitope positivity and were less spread on CTLD1 and/or CTLD7 epitope-containing domains. As for immunodominance, patients reaching remission were more often iCR or non-iDom than iC1, indicating that immunodominance is a novel predicting factor of clinical outcome.

### Anti-PLA2R1 Titer Predicts Clinical Outcome in Adjusted Analysis

Several previous studies have shown that anti-PLA2R1 titer predicts clinical outcome. Thus, before testing immunodominance as a novel predictive factor, the inventors validated that this could be observed in their cohort when considering the same population of 135 patients for which they had all the relevant data. In unadjusted analysis, anti-PLA2R1 titer was associated with clinical outcome when comparing patients with anti-PLA2R1 titer below and above the median (64.8 RU/mL, FIG. 6D). Patients with a titer above the median had worse clinical outcome (OR=0.237 [0.114-0.492], p=0.0001). In adjusted analysis, patients with titers above 64.8 RU/mL had a 4-fold lower chance to reach remission, independently from baseline eGFR and proteinuria levels as well as treatment (NIAT versus IS).

Considering the combination of anti-PLA2R1 titer and treatment, the inventors observed that, as compared to NIAT-treated patients with an anti-PLA2R1 titer below 64.8 RU/mL, patients who received the same conservative therapy but had anti-PLA2R1 titer above 64.8 RU/mL had a 5-fold lower chance to reach remission, independently from eGFR and proteinuria. Similarly, IS-treated patients with an anti-PLA2R1 titer below 64.8 RU/mL had a 3-fold higher chance to reach remission as compared to IS-treated patients with an anti-PLA2R1 titer above 64.8 RU/mL (OR=3.149 [1.142-8.686], p=0.03).

### Immunodominance Predicts Clinical Outcome in Adjusted Analysis

The inventors then tested whether the type of immunodominance can also predict clinical outcome in adjusted analysis. Since non-iDom patients had clinical and immunological characteristics more similar to iCR than iC1 patients and a similar chance of remission, the inventors compared immunodominance between iCR/non-iDom patients combined as a single group versus iC1 patients. In unadjusted analysis, immunodominance was associated with clinical outcome. In adjusted analysis, iC1 patients had about 3-fold lower chance to reach remission than iCR/non-iDom patients, independently from baseline eGFR and proteinuria levels as well as treatment (NIAT vs IS). When combining immunodominance and treatment, iCR/non-iDom patients treated with immunosuppressants had about 3-fold more chance to reach remission than NIAT-treated patients. Furthermore, iCR/non-iDom patients treated with immunosuppressants had 4.5-fold more chance to enter into remission than iC1 patients also treated with immunosuppressants (OR=4.467 [1.605-12.432], p=0.004,). Similar observations were made when iCR and non-iDom patients were considered as separate groups, yet with lower to barely significant p-values.

Considering the ratio of anti-CTLD1/anti-CysR titers as an alternative method to identify the immunodominant profile, the inventors also tested this method to predict clinical outcome. In unadjusted analysis, the median ratio of anti-CTLD1/anti-CysR titer was associated with clinical outcome (FIG. 9). In adjusted analysis, patients (n=134) with a ratio of anti-CTLD1/anti-CysR titers above the median (0.0324), thus mainly iC1) had about 3-fold lower chance to reach remission than patients with a titer ratio below the median (0.0324, thus mostly iCR), independently from baseline eGFR and proteinuria as well as treatment (NIAT vs IS). When combining the median ratio of titers and treatment, patients below the median (thus mainly iCR) and treated with immunosuppressants had 4-fold more chance to reach remission that patients above the median and treated with immunosuppressants (OR=3.956 [1.368-11.440], p=0.0196).

### Added Values Between Anti-Pla2r1 Titer And Immunodominance To Predict Clinical Outcome In Adjusted Analysis

To evaluate the added value of immunodominance over anti-PLA2R1 titer, the inventors performed adjusted analyses combining anti-PLA2R1 titer and other antibody features. Anti-PLA2R1 titer was significant to predict clinical outcome by itself (after adjustment for eGFR, proteinuria and treatment). Adjusted analyses for immunodominance remained significant, suggesting that immunodominance has an added value over anti-PLA2R1 titer. Specifically, patients with an iC1 immunodominant profile had a lower chance to reach remission independent of anti-PLA2R1 titer (OR=2.358 [1.076-5.155]).

Similarly, patients with a median ratio of anti-CTLD1/anti-CysR titers above the median (thus mostly iC1) had a lower chance to reach remission compared to patients below the median, independently of the anti-PLA2R1 titer (OD=2.831 [1.313-6.106]).

### Immunodominance Help to Refine the Likelihood of Response to Rituximab

Rituximab is becoming the first-line immunosuppressive therapy to treat severe MN. However, the likelihood of response to rituximab decreases in patients with high anti-PLA2R1 titer, above a certain cut-off ELISA value which is not yet clearly defined but might be around 200 RU/mL (Ruggenenti, P, Debiec, H, Ruggiero, B, Chianca, A, Pelle, T, Gaspari, F, Suardi, F, Gagliardini, E, Orisio, S, Benigni, A, Ronco, P, Remuzzi, G: Anti-Phospholipase A2 Receptor Antibody Titer Predicts Post-Rituximab Outcome of Membranous Nephropathy. J Am Soc Nephrol, 26: 2545-2558, 2015; De Vriese, AS, Glassock, RJ, Nath, KA, Sethi, S, Fervenza, FC: A Proposal for a Serology-Based Approach to Membranous Nephropathy. J Am Soc Nephrol, 28: 421-430, 2017).

Since rituximab was the main immunosuppressant given to patients in the study cohort, the inventors took advantage of this cohort to test whether immunodominance may have an added value to predict the likelihood of response to rituximab when anti-PLA2R1 titer is below 200 RU/mL.

First, the inventors performed a sensitivity analysis excluding patients treated with immunosuppressants other than rituximab (i.e. removing 20 patients) to determine whether immunodominance still predict clinical outcome. Results were similar to the whole cohort. On one hand, among iCR/non-iDom patients, those treated with rituximab had 4.5-fold higher chance to reach remission than NIAT-treated patients (OR=4.615 [1.486-14.329). On the other hand, among patients treated with rituximab, iCR/non-iDom patients had 6-fold higher chance to reach remission than iC1 patients, suggesting that iC1 patients are more resistant to treatment (OR=5.998 [1.771-20.311], data not shown). Together, this suggests that iCR/non-iDom patients have a higher chance to respond to rituximab than iC1 patients.

Second, considering the above lower response to rituximab for patients with high anti-PLA2R1 titer and the recent proposal for a serology-based approach of MN with 204 RU/mL as a possible upper cut-off value, the inventors selected patients with baseline anti-PLA2R1 titers below 200 RU/mL, who may need rituximab therapy and may have a better chance of response, and analyzed whether immunodominance might help to refine the likelihood of response to rituximab in this population. Below 200 RU/mL, 51.2% of patients were treated with rituximab while others (48.9%) were NIAT-treated. iCR/non-iDom and iC1 patients presented differences in proteinuria, but not in other clinical characteristics, including anti-PLA2R1 titer. Interestingly, iC1 patients had an overall worse clinical outcome compared to iCR/non-iDom patients (FIG. 6E), and among those treated with rituximab, a majority did not respond to treatment while most iCR patients responded and reached remission (61.5% in no remission for the iC1 group versus 89.7% in remission for the iCR/non-iDom group, FIG. 6E). Similar results were observed when considering the ratio of anti-CTLD1/anti-CysR titers to determine patients immunodominant profile (most right panel in FIG. 9B).

In conclusion, the combined evaluation of anti-PLA2R1 titer and immunodominance (assessed either by competition assay or by analysis of the ratio of anti-CTLD1/anti-CysR antibody titers) profile may help to better predict the likelihood of response to rituximab.

1. An in vitro method of predicting the prognosis of a patient suffering from membranous nephropathy comprising a step of determining the nature of the antibody which mostly drives the anti-PLA2R1 humoral response in a sample obtained from said patient, wherein:
if the anti-PLA2R1 humoral response in the patient’s sample is mostly driven by anti-CTLD 1 antibodies then the patient exhibits a poor prognosis.
if the anti-PLA2R1 humoral response in the patient’s sample is not mostly driven by anti-CTLD1 antibodies then the patient exhibits a good prognosis.

2. An in vitro method of predicting the response to an immunosuppressant of a patient suffering from membranous nephropathy comprising a step of determining the nature of the antibody which mostly drives the anti-PLA2R1 humoral response in a sample obtained from said patient, wherein:
if the anti-PLA2R1 humoral response in the patient’s sample is mostly driven by anti-CTLD 1 antibodies then the patient is a poor responder to the immunosuppressant;
if the anti-PLA2R1 humoral response in the patient’s sample is not mostly driven by anti-CTLD1 antibodies then the patient is a good responder to the immunosuppressant.

3. The method according to claim 1, wherein the step of determining PLA2R1 immunodominance comprises a step of performing a competition assay with saturating amounts of polypeptide competitors selected among a CysR fragment, a CTLD1 fragment, a CysR-FnII-CTLD1 fragment, or a mixture thereof.

4. The method according to claim 3, wherein the step of determining PLA2R1 immunodominance is performed by incubating the sample with saturating amounts of polypeptide competitors selected from a CysR fragment, a CTLD1 fragment, a CysR-FnII-CTLD1 fragment, a CTLD2-8 fragment or a mixture thereof.

5. The method according to any one of claim 4, wherein the sample is incubated with saturating amounts of CysR, CTLD1, CysR-FnII-CTLD1, CTLD7 and CTLD5 fragments.

6. The method according to any one of claim 1, wherein the step of determining the nature of the antibody which drives the anti-PLA2R1 response is achieved by establishing a ratio between the titers of anti-CTLD 1 antibodies and anti-CysR antibodies.

7. The method according to claims 6, wherein the measured value of the ratio is compared to a reference value.

8. The method of claim 2, wherein the immunosuppressant is rituximab.

9. The method according to claim 1, comprising a further step of determining the anti-PLA2R1 titer of the patient.

10. The method according to claim 9, wherein the anti-PLA2R1 titer of the sample is lower than 250 RU/mL.

11. The method according to claim 1, wherein the sample is selected from bodily fluids and derivatives which may or may not contain cells or biopsies.

12. The method according to claim 11, wherein the sample is whole blood, serum, plasma, urine or a kidney biopsy.

13. The method according to claim 12 wherein the sample is a serum or a urine sample.

14. The method according to claim 3, wherein,
when competition with a CysR-FnII-CTLD1 fragment is higher than 50% while competition with a CTLD1 fragment is lower than 30%, then the patient is immunodominant for CysR (iCR) and has a good prognosis and is a good responder to the immunosuppressant,
when competition with a CysR-FnII-CTLD1 fragment is higher than 50% while competition with a CTLD1 fragment is higher than 10% then the patient is immunodominant for CTLD1 (iC1) and has a bad prognosis and is a poor responder to the immunosuppressant, and
when competition with a CysR-FnII-CTLD1 fragment is lower than 50%, while competition with any other PLA2R1 domain is too low to determine a specific type of immunodominance, then the patient is non-immunodominant and has a good prognosis and is a good responder to the immunosuppressant.

15. A kit comprising:
Mmeans for detecting an autoantibody binding to CysR, CTLD1 or the extracellular domain of PLA2R1, and either
an immobilized polypeptide comprising CysR or a variant thereof binding to autoantibodies to CysR,
an immobilized polypeptide comprising CTLD 1 or a variant thereof binding to autoantibodies to CTLD1,
one or more immobilized polypeptides comprising CTLD5, CTLD7 and CTLD8 or a variant thereof binding to autoantibodies to CTLD5, CTLD7 or CTLD8, respectively,
an immobilized polypeptide comprising the extracellular domain of PLA2R1 or a variant thereof binding to autoantibodies to the extracellular domain of PLA2R1 and
optionally a negative control or cut-off indicator indicating non-specific binding of autoantibodies,
wherein the immobilized polypeptides are immobilized on one or more diagnostically useful carriers spatially separated such that an antibody bound to one of the polypeptides can be distinguished from an autoantibody bound to any of the other polypeptides; or
an immobilized polypeptide comprising the extracellular domain of PLA2R1 or a variant thereof binding to autoantibodies to the extracellular domain of PLA2R1,
a non-immobilized polypeptide comprising CysR or a variant thereof binding to autoantibodies to CysR_{,}
a non-immobilized polypeptide comprising CTLD1 or a variant thereof binding to autoantibodies to CTLD1,
one or more immobilized polypeptides comprising CTLD5, CTLD7 and CTLD8 or a variant thereof binding to autoantibodies to CTLD5, CTLD7 or CTLD8, respectively, and
optionally, a negative control or cut-off indicator indicating non-specific binding of autoantibodies
wherein the kit further comprises at least one control comprising an antibody to CysR and a second control comprising an antibody to CTLD1, and optionally in addition a control comprising an antibody to CTLD5, a control comprising an antibody to CTLD7, a control comprising an antibody to CTLD8, and optionally a control measuring non-specific binding.

16. The kit of claim 15, wherein the at least one₋control comprises
a first control comprising antibody to CysR and a second control comprising an antibody to CTLD1.

17. The kit of claim 16, further comprising one or more of: a control comprising an antibody to CTLD5, a control comprising an antibody to CTLD7 and a control comprising an antibody to CTLD8.

18. The method according to claim 10, wherein the anti-PLA2R1 titer of the sample is lower than 200 RU/mL.
